(19) United States
(12) Patent Application Publication
(10) Pub. No.: US 2025/0249226 A1
(43) Pub. Date: Aug. 7, 2025

(54) PLANAR MICRONEEDLE, MICRONEEDLE PATCH, MANUFACTURING DEVICE, STANDING DEVICE AND PREPARATION METHOD

(71) Applicants: YOUWE(ZHUHAI)BIOTECHNOLOGY CO., LTD, Guangdong (CN); CREWAY (ZHUHAI) PHARMACEUTICAL TECHNOLOGY CO., LTD, Guangdong (CN)

(72) Inventors: Chengguo LI, Guangdong (CN); Gang LENG, Guangdong (CN); Yonghao MA, Guangdong (CN); Jinyong CHEN, Guangdong (CN); Lianhua CHEN, Guangdong (CN); Yanfen LI, Guangdong (CN)

(21) Appl. No.: 18/856,599

(22) PCT Filed: Dec. 22, 2022
(86) PCT No.: PCT/CN2022/141169

§ 371 Date: Oct. 11, 2024

(30) Foreign Application Priority Data
Apr. 15, 2022 (CN) 202210399470.4
May 20, 2022 (CN) 202210556439.7
May 20, 2022 (CN) 202210556442.9

(51) Int. Cl. A61M 37/00

(52) U.S. Cl. CPC A61M 37/0015; A61M 2037/0046; A61M 2037/0053; A61M 2037/0061

(57) ABSTRACT

A planar microneedle includes a base layer, and microneedle(s) including a needle tip, a middle part, and a needle base. The microneedle further includes a first side-wall located on the same plane as the base layer, a circumferential side-wall, and a third side-wall, the first side-wall and the circumferential side-wall being located circumferentially around the microneedle, and the third side-wall being located at the bottom of the microneedle. The third side-wall includes a first edge connected with the base layer, and a movable edge. The microneedle is rotated around the first edge until an angle between the third side-wall and the plane where the base layer is located is 0°-±10°, and a base through-hole is formed at the junction of the microneedle and the base layer. A microneedle patch, a planar microneedle manufacturing device, a planar microneedle preparation method, and a planar microneedle standing device are provided.

### TECHNICAL FIELD

The present disclosure relates to the field of microneedle technology, specifically relating to a planar microneedle, a microneedle patch, a manufacturing device, a standing device, and a preparation method.

### BACKGROUND ART

The microneedle patch primarily includes a base layer and microneedles disposed thereon. During use, the side of the microneedle patch provided with microneedles is pressed against and adhered to the skin. Due to the relatively short length of the microneedles, it fulfills drug delivery requirements without causing nerve damage or pain, thereby gradually gaining public acceptance.

The microneedle patch is predominantly molded via a mold, wherein a drug-loaded solution is infused into the forming grooves of the mold via methods such as high-pressure filling or vacuum suction. This results in the direct formation of microneedles extending in a direction perpendicular to the base layer. To ensure that a patient is administered with the solution of a predetermined dosage during application of the microneedle patch, the microneedle's needle tip serves as the drug-loading area. Given the large depth and high aspect ratio of the forming grooves utilized for microneedle fabrication, the drug-loaded solution tends to disperse in a fan-shaped during filling. This dispersion leads to splashing of the drug-loaded solution onto portions of the forming grooves corresponding to the middle part and needle base of the formed microneedle. Alternatively, when employing vacuum suction, the drug-loaded solution traverses from portions of the forming grooves corresponding to the needle base and middle part of the formed microneedle to the needle tip, resulting in drug distribution throughout the needle tip, middle part, and needle base of the formed microneedle. The variability in penetration depths of the microneedles during application results in inconsistent drug dosages administered to the patient, thereby complicating precise dosage control. Additionally, the retention of drug-loaded solution within the forming grooves presents challenges in removal, leading to contamination that adversely affects subsequent batches of microneedles. Moreover, the considerable depth and aspect ratio of the forming grooves contribute to increased demolding resistance, thereby rendering the removal of the formed microneedles from the mold more difficult.

Accordingly, there exists an urgent need for a microneedle designed to concentrate the drug-loaded solution at the needle tip, as well as an associated manufacturing device and preparation method for forming the microneedle.

### SUMMARY

To address the aforementioned drawbacks and limitations of the prior art, the present disclosure provides a planar microneedle, a microneedle patch, standing and manufacturing devices and preparation method that are suited for using the microneedle patch formed by a two-step forming process. The process involves firstly forming a planar microneedle in which the first sidewall of the microneedle and the base layer are located in the same plane, and then a planar microneedle standing device adjusting the microneedle so that the angle between the third sidewall of the microneedle and the plane where the base layer is located is 0° to ±10°, thereby forming the microneedle patch. This disclosure effectively resolves technical issues encountered in conventional microneedle patch molding processes, specifically the challenges of difficult demolding, low precision in drug loading at the needle tip, and the complications associated with filling oil-water mixture raw material solutions or highly viscous raw material solutions into the forming grooves.

To achieve the aforementioned objectives, the present disclosure provides the following technical solutions.

A planar microneedle includes a base layer and at least one microneedle rotatably provided on the base layer. Each microneedle includes a needle tip, a needle base, and a middle part connecting the needle tip and the needle base. The direction of the needle tip towards the needle base is aligned with the extension direction of the base layer. The planar microneedle is fabricated through the process of filling and drying a raw material solution.

Specifically, the microneedle includes a first sidewall, a circumferential sidewall, and a third sidewall. The first sidewall and the circumferential sidewall are positioned circumferentially around the microneedle, while the third sidewall is located at the bottom of the microneedle. The first sidewall and the base layer reside in the same plane, with the third sidewall connected to the base layer. The microneedle is configured to rotate around the junction between the third sidewall and the base layer.

Specifically, the third sidewall comprises a first edge that connects to one side of the base layer away from the first sidewall. The microneedle is configured to rotate about the first edge, in the direction of the third sidewall towards the first sidewall until the angle between the third sidewall and the plane where the base layer is located is 0° to ±10° and a base through-hole is formed at the junction between the microneedle and the base layer.

Specifically, the microneedle is configured to rotate about the first edge until the third sidewall partially abuts against the side of the base layer close to the microneedle. The third sidewall includes a movable edge connected to the first edge, wherein the angle θ between the movable edge and the sidewall of the base through-hole is in a range of 0°<θ≤10°.

Specifically, the ratio of the maximum depth of the microneedle in the direction perpendicular to the base layer to the maximum length of the microneedle in the direction parallel to the base layer is 1:5 to 1:2.

A microneedle patch includes the aforementioned planar microneedle.

Specifically, the microneedle patch further includes an adhesion layer. The microneedle includes a first sidewall, a circumferential sidewall, and a third sidewall, with the first sidewall and the circumferential sidewall positioned circumferentially around the microneedle, while the third sidewall is located at the bottom of the microneedle. The first sidewall and the base layer are positioned in the same plane, with the third sidewall connected to the base layer. The microneedle is configured to rotate about the junction of the third sidewall and the base layer, such that both the microneedle and the side of the base layer away from the microneedle are adhered to the adhesion layer.

A planar microneedle manufacturing device for manufacturing the aforementioned planar microneedle includes a base, a mold, and a panel positioned above the base.

The top of the base is provided with a recess configured to accommodate the mold.

The top of the mold is provided with multiple recessed forming grooves used for forming microneedles.

The forming grooves each include a needle tip groove, a needle base groove, and a middle part groove. The middle part groove communicates with the needle tip groove and the needle base groove, with the direction of the needle tip groove towards the needle base groove being a horizontal direction.

The panel is provided with a forming hole configured to accommodate all of the forming grooves of the mold, wherein the forming hole is configured for molding the base layer.

The top of the mold protrudes upwards to form a separation portion, which is arranged circumferentially around the sidewall of the forming groove.

When the mold is fitted into the recess of the base, and the panel is pressed onto the tops of the mold and the base, the height of the separation portion is greater than or equal to the height of the panel.

Specifically, along the direction of the needle tip groove towards the needle base groove of the forming groove, the bottom wall of the forming groove extends outwards to form a protruding portion forming groove. The protruding portion forming groove is configured to mold the protruding portion on the third sidewall of the microneedle.

Specifically, when the lower edge of the forming-groove bottom wall of the forming groove rotates around the upper edge of the forming-groove bottom wall until the forming-groove bottom wall abuts against the top of the mold, the length of the lower edge is greater than the length of the line connecting the two intersection points of the lower edge with the forming groove.

A method for preparing a planar microneedle, using the aforementioned planar microneedle manufacturing device, includes the following steps:

A1: assembling the base, mold, and panel.

A2: filling the drug-loaded solution into the needle tip groove of the mold and drying to form the needle tip of the microneedle.

A3: filling the raw material solution into the middle part groove and the needle base groove of the mold, and the forming hole of the panel, and then scraping and leveling the raw material solution along the top of the panel.

The raw material solution within the forming hole dries to form the base layer, the raw material solution within the needle base groove of the mold dries to form the needle base of the microneedle, and the raw material solution within the middle part groove of the mold dries to form the middle part of the microneedle.

Specifically, step A3 further includes:
filling the raw material solution into the forming hole of the panel, and then scrapping and leveling the drug-loaded solution in the direction from the needle tip groove of the mold towards the needle base groove.

Alternatively, after filling the raw material solution into the forming hole of the panel until the forming hole is filled, the raw material solution is scraped and leveled along the top of the panel, followed by vacuuming.

Alternatively, after filling the raw material solution into the forming hole of the panel until the forming hole is filled, the raw material solution is scraped and leveled along the top of the panel, followed by centrifuging.

A planar microneedle standing device used for making the aforementioned planar microneedle stand up includes a planar microneedle conveying assembly and a planar microneedle rotating assembly.

The planar microneedle conveying assembly includes a planar microneedle conveyor belt, which is used to convey the planar microneedle to the planar microneedle rotating assembly.

The planar microneedle rotating assembly is used to apply pressure to the at least one microneedle of the planar microneedle, to make the at least one microneedle rotate around the junction between the third sidewall and the base layer, so as to make the microneedle to rotate from a configuration in which the first sidewall and the base layer are located in a same plane to a configuration in which the angle formed between the third sidewall and the plane where the base layer is located is 0° to ±10°, thereby accomplishing the standing of the at least one microneedle.

Specifically, the planar microneedle rotating assembly may be a vacuum negative pressure adhesive component, which is configured to apply a negative pressure airflow to the at least one microneedle from below the at least one microneedle.

Alternatively, the planar microneedle rotating assembly is a wind pressure adhesive component, the wind pressure adhesive component being used to apply airflow to the at least one microneedle from above the at least one microneedle.

Alternatively, the planar microneedle rotating assembly is a roller contact pressing component, which is configured to roll on the planar microneedle to apply pressure to the at least one microneedle.

Specifically, the planar microneedle rotating assembly may include a rotating mechanism and a microneedle accommodating mechanism. The rotating mechanism is a pressing plate.

The rotating mechanism is configured to apply pressure to the at least one microneedle, enabling the at least one microneedle to rotate around the first edge until the at least one microneedle is positioned within at least one accommodating groove located at the top of the microneedle accommodating mechanism, thereby completing the standing of the at least one microneedle.

Specifically, the bottom of the pressing plate is provided with multiple pressure application portions, which are arranged in one-to-one correspondence with multiple accommodating grooves.

Specifically, the longitudinal cross-section of each of the pressure application portions is in a downwardly protruding arc shape or a V shape.

Specifically, the planar microneedle rotating assembly may further include a rotating mechanism and a microneedle accommodating mechanism. The rotating mechanism includes a first roller, which is configured to apply pressure to the at least one microneedle to make the at least one microneedle rotate around the first edge until the at least one microneedle is positioned within at least one accommodating groove at the top of the microneedle accommodating mechanism, thereby completing standing of the at least one microneedle. The first roller and the microneedle accommodating mechanism may moving towards each other.

Specifically, the rotating mechanism further includes a second roller and a third roller.

The second roller is wrapped with an adhesion layer, and the second roller is used to convey the adhesion layer to the first roller. The third roller is used to be wrapped with and store a protective film that is obtained by tearing off the adhesion layer from the first roller. The first roller is used to apply pressure to the at least one microneedle and adhere the adhesion layer to the side of the base layer that is away from the at least one microneedle and to the at least one microneedle.

Specifically, a protective film peeling structure is provided on the side of the first roller close to the third roller. The protective film peeling structure is configured to control the position at which the protective film is peeled off from the adhesion base.

Specifically, the microneedle accommodating mechanism further includes multiple spaced fixing plate assemblies.

The fixing plate assembly includes a first clamping plate and a second clamping plate, wherein the first clamping plate is capable of moving in a direction towards or away from the second clamping plate.

When the first clamping plate abuts against the second clamping plate, the accommodating groove is formed between the first clamping plate and the second clamping plate.

When the microneedle is positioned within the accommodating groove, the first clamping plate and the second clamping plate clamp the microneedle.

Specifically, a limiting plate is provided at the end of the microneedle accommodating mechanism away from the planar microneedle conveying assembly, with the planar microneedle abutting against the limiting plate.

Specifically, the planar microneedle conveying assembly includes a planar microneedle conveyor belt and a standing conveyor belt. The planar microneedle conveyor belt is arranged inclinedly and is used to convey the planar microneedle. The standing conveyor belt is horizontally provided and located at the lower end of the planar microneedle conveyor belt. The standing conveyer belt is used to convey a viscous adhesion layer and receive the planar microneedle from the planar microneedle conveyor belt. The angle between the extension direction of the planar microneedle conveyor belt and the extension direction of the standing conveyor belt is 135 to 179°.

The beneficial effects of the present disclosure are as follows.

For the planar microneedle of the present disclosure, the first sidewall and the base layer are located within the same plane. Based on this microneedle film structure, within the forming groove of the mold used for molding the microneedle, the direction that the needle tip groove for forming the needle tip faces the needle base groove for forming the needle base is a horizontal direction. This reduces the depth of the forming groove and the ratio of the maximum depth to the maximum length of the forming groove, allowing for precise filling of the raw material solution into the needle tip groove of the forming groove. This is particularly suited for the infusion of oil-water mixtures or high-viscosity raw material solution, while preventing the raw material solution from splashing and contaminating of the middle part groove and needle base groove. Consequently, the formed microneedle carries the drug only at the needle tip, enabling precise control of the dosage administered during use. Furthermore, by reducing the depth of the forming groove and the ratio of maximum depth to maximum length, the demolding resistance between the formed microneedles and the forming groove can be further reduced, thereby preventing microneedle breakage, improving the yield of the microneedles, and lowering production costs.

### BRIEF DESCRIPTION OF DRAWINGS

FIG. 1 is a perspective schematic view of a planar microneedle according to Embodiment 1 of the present disclosure;

FIG. 2 is a perspective schematic view of a microneedle patch according to Embodiment 2 of the present disclosure (the microneedle is tetrahedral in shape);

FIG. 3 is a schematic view of FIG. 2 from another viewing angle;

FIG. 4 is a partial enlarged schematic view of FIG. 3;

FIG. 5 is a perspective schematic view of the microneedle patch according to Embodiment 2 of the present disclosure (the microneedle is in the shape of tetrahedrons and the third sidewall of the microneedle is provided with a protruding portion);

FIG. 6 is a partial enlarged schematic view of FIG. 5;

FIG. 7 is a perspective schematic view of the microneedle patch according to Embodiment 2 of the present disclosure (the microneedle is in the shape of pentagonal pyramids and the third sidewall of the microneedle is provided with a protruding portion);

FIG. 8 is a perspective schematic view of the microneedle patch according to Embodiment 2 of the present disclosure (the microneedle is in the shape of circular cone or elliptical cone);

FIG. 9 is a perspective schematic view of the microneedle patch according to Embodiment 2 of the present disclosure (the microneedle is in the shape of triangular pyramids and both sides of the first sidewall of the microneedle are provided with protrusions);

FIG. 10 is a perspective schematic view of the microneedle patch according to Embodiment 2 of the present disclosure (the microneedle is in the shape of tetrahedrons and both sides of the first sidewall and the second sidewall of the microneedle are all provided with protrusions);

FIG. 11 is a perspective schematic view of the microneedle patch according to Embodiment 2 of the present disclosure (the microneedle is in the shape of tetrahedrons, with protrusions provided on both sides of the first sidewall and on the second sidewall, and a protruding portion on the third sidewall of the microneedle);

FIG. 12 is an exploded-view of the planar microneedle forming device according to Embodiment 3 of the present disclosure;

FIG. 13 is an assembly view of FIG. 12;

FIG. 14 is an exploded-view of the mold and the planar microneedle in FIG. 12;

FIG. 15 is a perspective schematic view of the mold shown in FIG. 12 (the length of the lower edge of the bottom wall of the forming groove of the mold is greater than the length of the upper edge);

FIG. 16 is a perspective schematic view of the mold shown in FIG. 12 (the bottom wall of the forming groove of the mode is provided with a protruding portion forming groove);

FIG. 17 is a schematic view from another perspective of the forming groove in FIG. 16.

FIG. 18 is a working flowchart of the planar microneedle standing device according to Embodiment 5 of the present disclosure (the rotating mechanism is a pressing plate).

FIG. 19 is a working flowchart of the planar microneedle rotating assembly in FIG. 18;

FIG. 20 is a partial enlarged view of FIG. 19 (before the microneedles stand up);

FIG. 21 is a partial enlarged view of FIG. 19 (after the microneedles stand up);

FIG. 22 is a perspective schematic view of FIG. 18;

FIG. 23 is a partial enlarged view of FIG. 22 (before the microneedles stand up);

FIG. 24 is a partial enlarged view of FIG. 22 (after the microneedles stand up);

FIG. 25 is a schematic view of the process of making the microneedle stand up, using a pressing plate;

FIG. 26 is a schematic view illustrating the pressure application portion of the pressing plate in FIG. 25 (the longitudinal cross-section of the pressure application portion is in a downwardly protruding arc shape);

FIG. 27 is a schematic view illustrating the pressure application portion of the pressing plate in FIG. 25 (the longitudinal cross-section of the pressure application portion is in a downwardly protruding V shape);

FIG. 28 is a working flowchart of the planar microneedle standing device according to Embodiment 5 of the present disclosure (the rotating mechanism is a roller assembly);

FIG. 29 is a partial enlarged view of FIG. 28;

FIG. 30 is a perspective schematic view of FIG. 28;

FIG. 31 is a partial enlarged view of FIG. 30 (the microneedles in the shape of tetrahedrons, and after standing, the third edge of the third wall of the microneedle abuts against the side of the base layer close to the microneedle);

FIG. 32 is a partial enlarged view of FIG. 30 (the microneedle is in the shape of triangular pyramids, and a protrusion structure is provided);

FIG. 33 is a partial view of the planar microneedle conveying assembly;

FIG. 34 is a perspective view of the microneedle patch (provided with an adhesion layer) of the present disclosure;

FIG. 35 is a front view of the planar microneedle standing device, employing a vacuum negative pressure adhesive component as the microneedle rotating assembly;

FIG. 36 is an enlarged view of Part D IN FIG. 35;

FIG. 37 is a perspective schematic view of FIG. 35;

FIG. 38 is a front view of the planar microneedle standing device, utilizing an wind pressure adhesive component as the microneedle rotating assembly;

FIG. 39 is an enlarged view of Part E in FIG. 38;

FIG. 40 is a perspective schematic view of FIG. 38;

FIG. 41 is a front view of the planar microneedle standing device, employing a roller contact pressing component as the microneedle rotating assembly;

FIG. 42 is an enlarged view of Part F in FIG. 41; and

FIG. 43 is a perspective schematic view of FIG. 41.

### REFERENCE NUMERALS

1. planar microneedle; 11. base layer; 111. gap; 112. base through-hole 12. microneedle; 121. first sidewall; 122. second sidewall; 123. third sidewall; 1231. first edge; 1232. second edge; 1233. third edge; 1234. arcuate edge; 124. needle tip; 125. middle part; 126. needle base; 127. protruding portion; 128. protrusion structure; 129. arcuate sidewall;
2. microneedle patch; 21. adhesion layer;
31. base; 311. recess; 32. mold; 321. forming groove; 3211. forming-groove bottom wall; 3212. needle tip groove; 3213. middle part groove; 3214. needle base groove; a. upper edge of forming-groove bottom wall; b. lower edge of forming-groove bottom wall; 3215. protruding portion forming groove; 3216. forming-groove sidewall; 322. separation portion; 323. first protrusion-fabricating recess; 33. panel; 331. forming hole;
41. planar microneedle conveying assembly; 4111. planar microneedle conveyor belt; 4112. standing conveyor belt; 412. storage device; 4121. discharge port; 413. planar microneedle adsorber; 414. blocking plate; 4141. first blocking plate; 4142. second blocking plate; 42. planar microneedle rotating assembly; 4211. pressing plate; 42111. pressure application portion; 4212. first roller; 4213. second roller; 4214. third roller; 422. microneedle accommodating mechanism; 4221. accommodating groove; 4222, the first clamping plate; 4223, the second clamping plate; 4224. limiting plate; 4225, top wall; 423. vacuum negative pressure adhesive component; 424. wind pressure adhesive component; 425. roller contact pressing component; 4251. roller; 4252. supporting stand.
A. B. two endpoints of the projection edge of the third edge being projected on the base through-hole, when the microneedle stands up and the third sidewall of the microneedle is parallel to the base layer.
F. G. two connection points of the third edge and second edge of the third sidewall.
H. I. endpoints of two ends of the first edge of the third sidewall.
J. K. two intersection points of the arcuate edge of the third side with the base through-hole.
C. D. two intersection points of the lower edge with the forming groove, when the forming-groove bottom wall is rotated about the upper edge until the forming-groove bottom wall is parallel to the top wall of the mold.
θ. angle between the second edge adjacent to the first edge and the sidewall of the base through-hole, when the third edge abuts against the side of the base layer near the microneedle.
L1. dimension of the third sidewall.
L2. dimension of the projection of the third sidewall being projected onto the adhesion laver.

### DETAILED DESCRIPTION OF EMBODIMENTS

The following describes the implementation of the present disclosure in conjunction with specific examples. Those skilled in the art will easily understand other advantages and effects of the present disclosure from the content disclosed in this description. The present disclosure may also be implemented or applied through other different specific Embodiments. The various details in this description may be modified or changed based on different perspectives and applications without departing from the spirit of the present disclosure.

Before further describing the specific embodiments of the present disclosure, it should be understood that the protection scope of the present disclosure is not limited to the following specific embodiments; it should also be understood that the terms used in the embodiments of the present disclosure are for describing specific embodiments, and not intended to limit the protection scope of the present disclosure.

### Embodiment 1

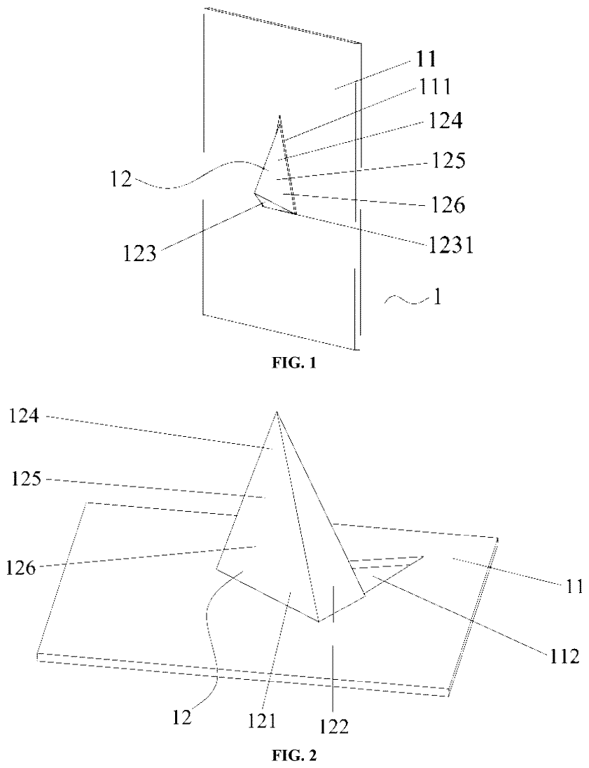

With reference to FIG. 1, this Embodiment provides a planar microneedle, which includes a base layer 11 and a microneedle 12, with the microneedle 12 being provided on the base layer 11. The microneedle 12 includes a needle tip 124, a middle part 125, and a needle base 126, where the middle part 125 connects the needle tip 124 and the needle base 126. The direction of the needle tip 124 towards the needle base 126 is the substantially same as the extension direction of the base layer 11. The microneedle 12 is formed in a forming groove 321 of a mold 32.

With reference to FIG. 14, on one hand, the direction in which the needle tip groove 3212 used for forming the needle tip 124 faces the needle base groove 3214 used for forming the needle base 126 is substantially horizontal direction, which reduces the depth of the forming groove 321 and decreases the ratio of the maximum depth to maximum length of the forming groove 321, facilitating the precise filling of the raw material solution into the needle tip groove 3212 of the forming groove 321, regardless of the type of the raw material solution. Since the forming groove 321 is arranged horizontally, when filling the raw material solution into the needle tip groove 3212, it is not required to pass through the middle part groove 3213 and the needle base groove 3214; instead, the raw material solution can be directly filled into the needle tip groove 3212, thus preventing the raw material solution from splashing and contaminating the middle part groove 3213 and the needle base groove 3214, ensuring that the formed microneedle 12 carries the drug only at the needle tip 124, thereby allowing precise control of the dosage delivered by the microneedle patch 2 during use. Additionally, reducing the ratio of the maximum depth to the maximum length of the forming groove 321 can also decrease the demolding resistance between the formed microneedle 12 and the forming groove 321, thus preventing the microneedle 12 from breaking, increasing the yield of the microneedle 12, and reducing costs. On the other hand, due to the forming groove 321 being horizontally provided, the dimensions of the needle tip groove 3212 can be larger than those of the middle part groove 3213 and/or the needle base groove 3214 in the horizontal direction, allowing the microneedle 12 to form protrusion structures 128 and other structures that prevent it from being detached from the skin, such as arrow-shaped, pinecone-shaped, half gourd-shaped structures, and facilitating the demolding of the microneedle 12.

Furthermore, the ratio of the maximum width to the maximum height of the microneedle 12 is between 1:5 to 1:2. Correspondingly, the ratio of the maximum depth to the maximum length of the forming groove 321 for forming the microneedle 12 is also between 1:5˜1:2. When the ratio of the maximum height to the maximum length of the microneedle 12 exceeds 5, the microneedle 12 becomes too sharp, making it prone to breakage when penetrating the skin. Additionally, if the microneedle 12 is too long, it may cause a significant sensation of pain upon insertion, leading to discomfort for the user.

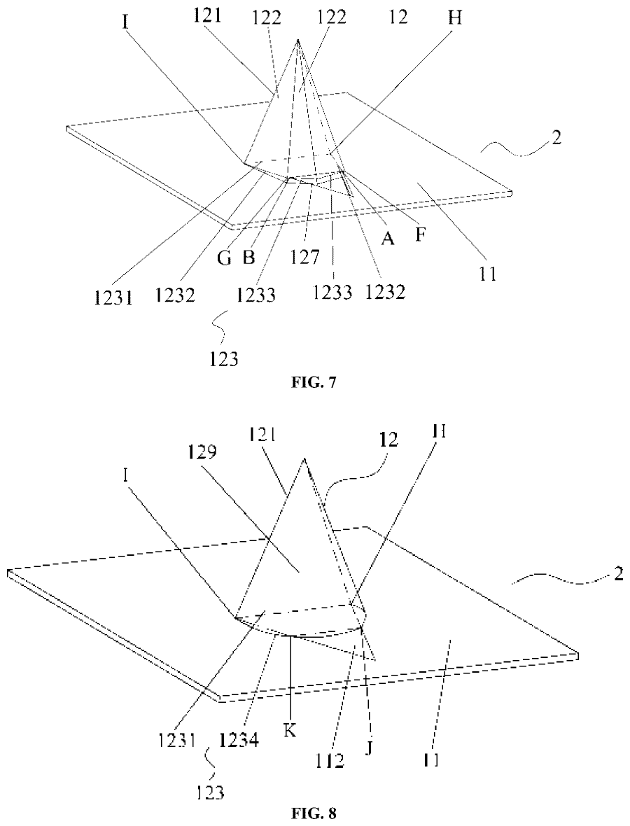

With reference to FIGS. 1-11, further, the microneedle 12 is in a conical shape. The microneedle 12 is in a pyramidal, circular cone, or elliptical cone shape.

Specifically, when the microneedle 12 is in the pyramidal shape, the microneedle 12 includes a first sidewall 121, a circumferential sidewall, and a third sidewall 123. The first sidewall 121 lies in the same plane as the base layer 11, with both the first sidewall 121 and the circumferential sidewall positioned circumferentially around the microneedle 12. The third sidewall 123 is located at the bottom of the microneedle 12. There is a gap 111 between the junction of the first sidewall 121 and the circumferential sidewall, and the base layer 11. When the microneedle 12 is in a pyramidal shape, the circumferential sidewall includes at least two second sidewalls 122. When the microneedle 12 is in the circular cone or elliptical cone shape, the circumferential sidewall includes an arcuate sidewall 129.

The planar microneedle 1 needs to be made stand up before being used to penetrate the skin to deliver the drug into the skin. The standing process of microneedle 12 includes that: the microneedle 12 rotates around the first edge 1231 and passes through the gap 111, where the microneedle 12 rotates from the state in which the first sidewall 121 of the microneedle 12 is coplanar with the base layer 11 (i.e, they are located in the same plane) to the state where the third sidewall 123 is positioned to be generally coplanar with or partially abuts against the base layer 11, such that the angle between the third sidewall 123 and the plane where the base layer 11 is located is 0° to ±10° (when the third sidewall 123 is inclined relative to the base layer 11 and positioned below the base layer 11, the angle between the third sidewall 123 and the plane where the base layer 11 is located is greater than or equal to −10° and less than 0°; and when the third sidewall 123 is inclined relative to the base layer 11 and positioned above the base layer 11, the angle between the third sidewall 123 and the plane where the base layer 11 is located is greater than 0° and less than or equal to 10°) , completing the standing of the microneedle 12 which is prepared into the microneedle patch 2. At this time, a base through-hole 112 is formed at the junction between the microneedle 12 and the base layer 11. When the third sidewall 123 is located within the base through-hole 112 and substantially parallel to the base layer 11 or the third sidewall 123 partially abuts against the base layer 11, the angle between the third sidewall 123 and the plane where the base layer 11 is located is 0°.

Preferably, after the microneedle 12 stands up, the direction of the needle tip 124 of the microneedle 12 towards the needle base 126 is either perpendicular to or intersects with the base layer 11. When using the microneedle patch 2, the direction of pressing the microneedle patch 2 is basically aligned with the direction of the needle base 126 towards the needle tip 124, such that with the application of minimal force, the microneedle 12 can penetrate the skin smoothly. At this time, the lateral shear force experienced by the microneedle 12 is nearly zero, effectively preventing the microneedle 12 from breaking during use of the microneedle patch 2.

Preferably, when the microneedle 12 is in the pyramidal shape:
the angle β between the first sidewall 121 and the third sidewall 123 is 45° to 90°. This angular configuration affects the difficulty of demolding process after molding of the microneedle 12 and the use performance of the microneedle patch 2. When β exceeds 90°, the demolding of the microneedle 12 after the molding becomes impractical. When β is less than 45°, the resulting angle between the first sidewall 121 of the microneedle 12 and the base layer 11 is small after the microneedle 12 stands up, resulting in a small inclination angle between the direction of the needle tip 124 of the microneedle 12 towards the needle base 126 and the base layer 11, such that during application, the microneedle 12 may be unable to penetrate the skin or that the resistance when the microneedle penetrates the skin is too large, leading to breakage of the microneedle 12, thereby affecting the normal use of the microneedle patch 2.

The angle α between the second sidewall 122 and the third sidewall 123 is 60° to 90°. This angle affects the volume and shape of the microneedle 12. When α is less than 60°, the width of the needle base 126 becomes relatively large. As a result, during the use of the microneedle patch 2, the increased width of the needle base 126 may lead to the needle base 126 remaining within the stratum corneum and failing to penetrate the target site. This affects the drug delivery stability of the microneedle 12 after penetration into the skin and results in waste of the forming material used at the needle base 126. When a exceeds 90°, the angle between the second sidewall 122 and the third sidewall 123 of the microneedle 12 becomes larger, resulting in a small height of the microneedle 12 once it stands up. Additionally, the inclination angle between the direction of the needle tip 124 towards the needle base 126 and the base layer 11 becomes greater. During the application of the microneedle patch 2, the angle between the direction of external force and the direction of the needle tip 124 towards the needle base 126 is large, causing the microneedle 12 to fail to penetrate the skin, or due to the higher insertion resistance, leading to the breakage of the microneedle 12, thereby affecting the normal use of the microneedle patch 2.

Further, the third sidewall 123 includes a first edge 1231 and a movable edge, where the first edge 1231 is connected to the base layer 11.

When the microneedle 12 is in the pyramidal, the movable edge includes at least one second edge 1232 and at least one third edge 1233. The first edge 1231 is connected to the base layer 11, and the first edge 1231 and at least one third edge 1233 are provided oppositely. The second edge 1232 connects the first edge 1231 and the third edge 1233.

For example, when the microneedle 12 is in the shape of a triangular pyramid, the third sidewall 123 includes the first edge 1231, the second edge 1232, and the third edge 1233. When the microneedle 12 is in the shape of rectangular pyramid, the third sidewall 123 includes one first edge 1231, two second edges 1232, and one third edge 1233. The first edge 1231 and the third edge 1233 are arranged in opposition to each other, and the two second edges 1232 are arranged oppositely, and each connect the first edge 1231 and the third edge 1233. When the microneedle 12 is in the shape of a pentagonal pyramid, the third sidewall 123 includes one first edge 1231. two second edges 1232, and two third edges 1233. The first edge 1231 and the two third edges 1233 are provided oppositely, and the two second edges 1232 are provided oppositely and each connect the first edge 1231 and the third edge 1233. Similarly, when the number of edges of the microneedle 12 is greater than or equal to four, the first edge 1231 and at least one of the third edges 1233 of the third sidewall 123 are provided oppositely, and the second edge 1232 connects the first edge 1231 and the third edge 1233.

Preferably, when the number of edges of the microneedle 12 is greater than or equal to four, the movable edge includes at least two second edges 1232 and at least one third edge 1233. The first edge 1231 is connected to the base layer 11, with the first edge 1231 and the third edge 1233 being provided oppositely. The second edge 1232 connects the first edge 1231 and the third edge 1233. When the microneedle 12 stands up, the microneedle 12 may rotate around the first edge 1231, from the state where the first sidewall 121 and the base layer 11 are located in the same plane to the state where the third edge 1233 of the third sidewall 123 abuts against the side of the base layer 11 close to the microneedle 12.

After the microneedle 12 stands up, the third sidewall 123 of the microneedle 12 partially abuts against the base layer 11. The length of the line connecting the two connection points FG of the third edge 1233 and the second edge 1232 of the third sidewall 123 is greater than the length of the projection edge AB of this line being projected on the base through-hole 112, allowing the third edge 1233 of the third sidewall 123 of the microneedle 12 to abut against the base layer 11. At this time, the third sidewall 123 forms a polygonal contact surface on the base layer 11 (the contact surface being enclosed by the HAF or IGB on the base layer 11), thereby ensuring that the third sidewall 123 stably abuts against the base layer 11. In other words, after the microneedle 12 rotates around the first edge 1231 and passes through the base through-hole 112 to complete the standing, the area of the third sidewall 123 is larger than the area of the base through-hole 112 corresponding to the third sidewall 123, allowing the third sidewall 123 to partially abut against the base layer 11, thereby being supported on the base layer 11, achieving the stable standing of the microneedle 12, and preventing the microneedle 12 from rebounding and rotating in the opposite direction around the first edge 1231 after the rotating force applied to the microneedle 12 is removed.

More preferably, the length of the line connecting the connection points FG of the third edge 1233 and the second edge 1232 of the third sidewall 123 is less than or equal to the length of the first edge 1231 (that is the length of HI), which facilitates the demolding of the microneedle 12 after the molding.

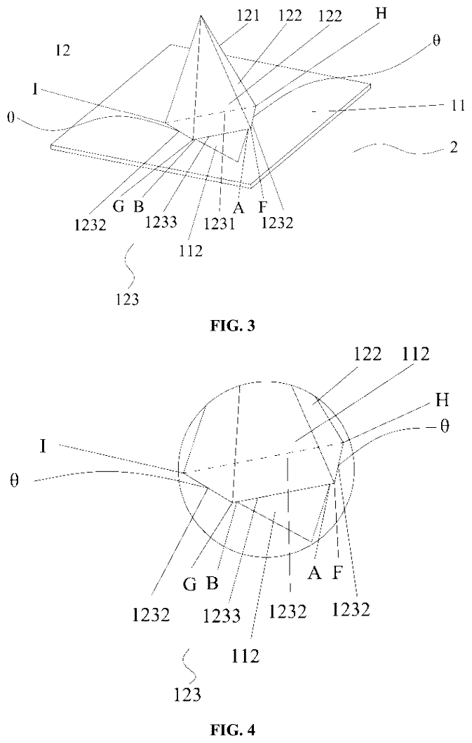

Referring to FIGS. 3-4, it is preferable that when the third edge 1233 of the third sidewall 123 of the microneedle 12 abuts the side of the base layer 11 near the microneedle 12, the angle θ between the second edge 1232 adjacent to the first edge 1231 and the sidewall of the base through-hole 112 is in the range of 0°<θ≤10°. When θ is greater than 10°, this may lead to an increased resistance to the rotation of the microneedle 12, resulting in damage to the microneedle 12 during standing. More preferably, 1°<θ≤6°.

When the microneedle 12 is in the shape of a circular cone or an elliptical cone, the movable edge includes an arcuate edge 1234.

The microneedle 12 is capable of rotating around the first edge 1231, from the state where the first sidewall 121 and the base layer 11 are located in the same plane to the state where the arcuate edge 1234 of the third sidewall 123 abuts against the side of the base layer 11 close to the microneedle 12.

Specifically, when the arcuate edge 1234 of the third sidewall 123 abuts against the side of the base layer 11 close to the microneedle 12, the arcuate edge 1234 intersects the base through-hole 112 at points J and K, allowing a contact surface to formed between the third sidewall 123 and the base layer 11, thereby making the third sidewall 123 stably abut against the base layer 11. In other words, after the microneedle 12 rotates around the first edge 1231 and passes through the base through-hole 112 to achieve the standing, the area of the third sidewall 123 is greater than the area of the base through-hole 112 corresponding to the third sidewall 123, allowing the third sidewall 123 to partially abut against the base layer 11 to be supported on the base layer 11, enabling stable standing of the microneedle 12, and preventing the microneedle 12 from rebounding and rotating in the opposite direction around the first edge 1231 after the rotating force applied to the microneedle 12 is removed.

After the microneedle 12 stands up, the arrangement of the third edge 1233 or the arcuate edge 1234 of the third sidewall 123 abutting against the base layer 11 can increase the gripping force between the microneedle 12 and the base layer 11, ensuring that the third sidewall 123 of the microneedle 12 stably abuts against the base layer 11. Therefore, it prevents the formation of a large angle between the third sidewall 123 and the base layer 11 of the microneedle 12 due to rebounding of the microneedle 12 after the rotating force applied onto the microneedle 12 is removed, which results in defects when adhering to the adhesion layer 21 or increase resistance during the microneedle 12 penetrating the skin and affects the normal use of the microneedle patch 2. At the same time, when the arcuate edge 1234 or the third edge 1233 of the microneedle 12 standing up abuts against the base layer 11, a contact surface is formed between the third sidewall 123 and the base layer 11. This contact surface provides supporting force for making the microneedle 12 stands up on the base layer 11. Consequently, the stable standing of the microneedle 12 on the base layer 11 may need no providing of adhesion layer 21 to assist in making the microneedle 12 stand up, on the microneedle 12 and the base layer 11. This microneedle patch 2, which does not include an adhesion layer 21, is suitable for use by individuals who are allergic to the material of the adhesion layer 21.

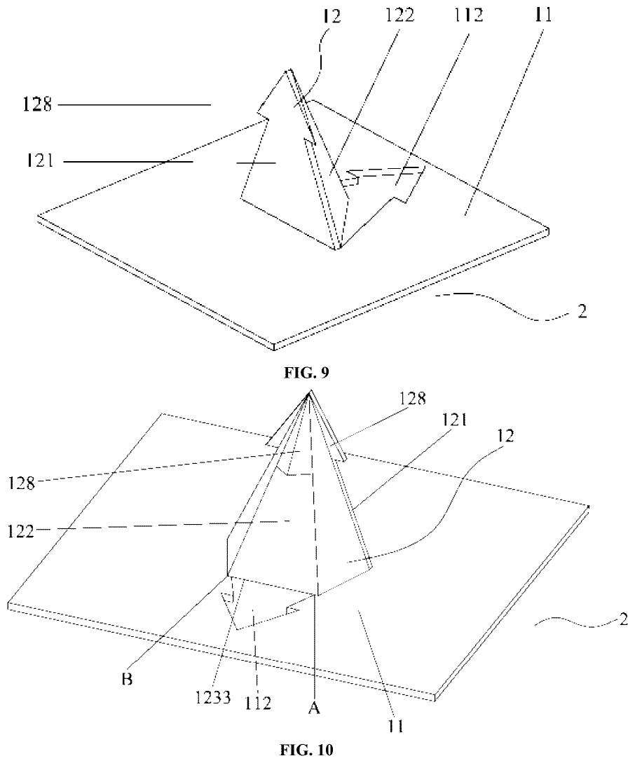

Referring to FIG. 9, further, protrusion structures 128 are arranged on two sides of the first sidewall 121 of the microneedle 12. These protrusion structures 128 extend in the direction from the needle tip 124 towards the needle base 126, forming a barbed configuration, with the protrusion structures 128 located at the needle tip 124. Through the protrusion structures 128 provided on the microneedle 12, the protrusion structures 128 may perform secondary penetration into the skin under the influence of the skin's elastic force after the microneedle 12 has been inserted into the skin, enhancing the gripping force between the microneedle 12 and the skin, preventing the microneedle 12 from being expelled due to the skin's elastic force. Furthermore, due to the sufficiently strong gripping force between the microneedle 12 and the skin, it can eliminate the need for an adhesion layer 21 for being adhered to the skin on the microneedle patch 2, making it suitable for use by individuals sensitive to adhesive material.

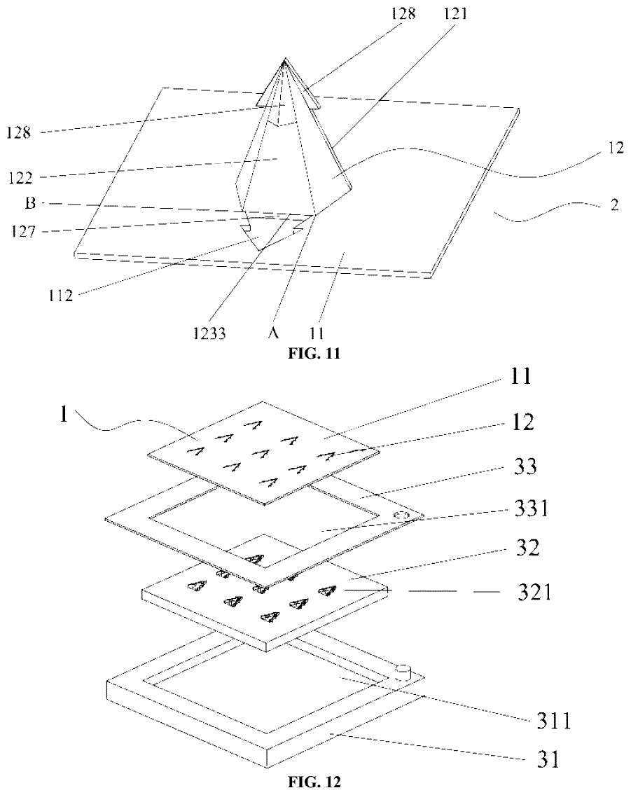

Referring to FIGS. 10 and 11, preferably, when the microneedle 12 is in a pyramidal shape and the number of the edges of the microneedle 12 is equal to or greater than four, a protrusion structure 128 is provided on the second sidewall 122, which is opposite to the first sidewall 121. The protrusion structure 128 extends in the direction from the needle tip 124 towards the needle base 126, forming a barbed configuration, with the protrusion structure 128 preferably located at the needle tip 124. When the microneedle 12 is in the shape of a circular cone or an elliptical cone, the arcuate sidewall 129 is provided with the protrusion structure 128. This protrusion structure 128 extends in the direction from the needle tip 124 towards the needle base 126, forming a barbed configuration, with the protrusion structure 128 preferably located at the needle tip 124.

By providing the protrusion structure 128 on the second sidewall 122 or the arcuate sidewall 129 and correspondingly providing a second protrusion-fabricating recess in the forming groove 321 of the mold 32 for forming of the protrusion structure 128, the raw material solution can be precisely filled into the second protrusion-fabricating recess during the formation of the microneedle 12, thereby enabling accurate control over the drug loading of the resulting microneedle 12. More preferably, the second sidewall 122 or the arcuate sidewall 129 is provided with multiple protrusion structures 128, and multiple second protrusion-fabricating recesses for forming the multiple protrusion structures 128 are correspondingly provided in the forming groove 321 of the mold 32, such that different raw material solution are separately filled into the multiple second protrusion-fabricating recesses in the forming groove 321 during the formation of the microneedle 12, allowing for precise control of the drug loading of the resulting microneedle 12 and enabling the formed microneedle 12 to carry multiple types of drugs.

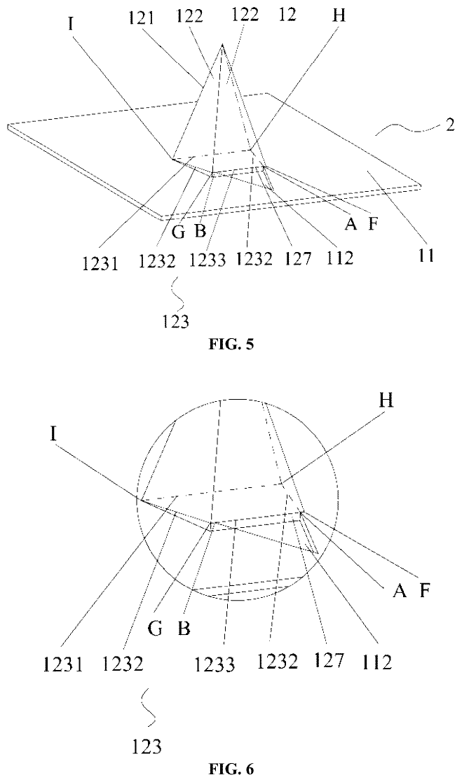

With reference to FIGS. 5-7, further, the third sidewall 123 extends outwards in the direction from the needle tip 124 towards the needle base 126 to form a protruding portion 127. This protruding portion 127 is capable of passing through the base through-hole 112.

During the standing of the microneedle 12, a planar microneedle 1 provided with no protruding portion 127 requires the microneedle 12 to be rotated until a large angle is formed between the third sidewall 123 of the microneedle 12 and the side of the base layer 11 that is close to the microneedle 12. This necessitates a greater rotating force, increasing the difficulty in material selection and type/model selection for the rotating mechanism that applies the rotating force to the microneedle 12. In contrast, a planar microneedle 1 provided with the protruding portion 127 only requires the microneedle 12 to be rotated until the protruding portion 127 is flush with the side of the base layer 11 that is away from the microneedle 12. At this point, the angle between the third sidewall 123 of the microneedle 12 and the side of the base layer 11 close to the microneedle 12 is small, decreasing the rotating force applied to the microneedle 12, facilitating the standing of the microneedle 12 and decreasing the difficulty in the material selection and type/model selection for the rotating mechanism used to apply the rotating force to the microneedle 12.

It is preferable that the maximum height of the protruding portion 127 is greater than or equal to the thickness of the base layer 11, such that when the third edge 1233 of the third sidewall 123 of the microneedle 12 abuts against the side of the base layer 11 that is close to the microneedle 12, the protruding portion 127 is flush with the side of the base layer 11 that is away from the microneedle 12 or the protruding portion 127 extends beyond the side of the base layer 11 away from the microneedle 12. When the maximum height of the protruding portion 127 is less than the thickness of the base layer 11, if the microneedle 12 is rotated until the protruding portion 127 is flush with the side of the base layer 11 that is away from the microneedle 12, it may easily lead to that the third sidewall 123 of the microneedle 12 does not penetrate or not completely penetrate the base layer 11, and result in the third edge 1233 of the microneedle 12 being unable to abut against or stably abut against the side of the base layer 11 that is close to the microneedle 12 to avoid that during standing of the microneedle 12, after the rotating force applied to the microneedle 12 is removed, the microneedle rebounds to cause the third sidewall 123 of the microneedle 12 to tilt relative to the base layer 11.

It is more preferable that the maximum height of the protruding portion 127 is equal to the thickness of the base layer 11. When the maximum height of the protruding portion 127 exceeds the thickness of the base layer 11, the protruding portion 127 extends beyond the side of the base layer 11 that is away from the microneedle 12 after the standing of the microneedle 12. Due to that the side of the base layer 11 away from the microneedle 12 is not a smooth surface, when forming the microneedle patch 2, the comfort of use of the microneedle patch 2 and the aesthetic appearance of the microneedle patch 2 may be affected easily. Furthermore, when applying the adhesion layer 21 to the protruding portion 127 of the microneedle 12, the adhesion layer 21 may be uneven due to that the protruding portion 127 extends beyond the side of the base layer 11 that is away from the microneedle 12.

### Embodiment 2

With reference to FIGS. 2-11 and 34, based on Embodiment 1, the present embodiment provides a microneedle patch. Specifically, it includes the following two types.

Referring to FIG. 34, when the microneedle patch 2 is provided with an adhesion layer 21: after the standing of the microneedle 12, the side of the base layer 11 away from the microneedle 12, as well as the third sidewall 123 or the protruding portion 127 of the microneedle 12, adhere to the adhesion layer 21, forming a microneedle patch 2 with the adhesion layer 21. This microneedle patch 2 can stably adhere to the skin surface through the adhesion layer 21 exposed via the base through-hole 112 once the microneedle 12 penetrates the skin. This prevents the microneedle 12 from being pushed out due to the skin's elastic force to affect the normal use of the microneedle patch 2.

When the microneedle patch 2 is not provided with an adhesion layer 21, the microneedle 12 contains components that are soluble in water easily, such as sodium hyaluronate. After the standing of the microneedle 12, it is thereby formed into a microneedle patch 2 without the adhesion layer 21.

When using the microneedle patch 2 without an adhesion layer 21, first, the needle tip 124 of the microneedle 12 is made face the skin, then the microneedle patch 2 is pressed until the microneedle 12 penetrates the skin. Purified water is applied to the base layer 11 to dissolve the base layer 11 and the portion of the microneedle 12 that has not entered the skin. Subsequently, the skin rebounds and closes the through-hole formed when the microneedle 12 penetrates the skin, encapsulating the microneedle 12 within the skin to enable dissolution and drug delivery. Since the base layer 11 contains sodium hyaluronate, when purified water dissolves the base layer 11, the sodium hyaluronate in the base layer 11 transforms into a solution, forming a protective film on the skin surface to reduce water loss from the skin surface, thereby accelerating the dissolution of the microneedle 12 within the skin. Furthermore, this type of microneedle patch 2 provided with no adhesion layer 21 is also suitable for use by children, other special patients, or pets. It can prevent situations where children or other special patients, or pets, might scratch at the adhesion layer 21 during use of the microneedle patch 2, causing the microneedle 12 to detach along with the adhesion layer 21 and thereby fail to achieve the drug delivery of the microneedle 12.

Preferably, a protrusion structure 128 is arranged on the microneedle 12. When the microneedle 12 is subjected to compressive rebound force from the skin, the protrusion structure 128 on the microneedle 12 enable secondary penetration into the skin, increasing the gripping force between the microneedle 12 and the skin, thereby preventing the skin from expelling the microneedle 12. More preferably, multiple protrusion structures 128 are staggered on two sides of the first sidewall 121. Compared to symmetrically arranged protrusion structures 128 on two sides of the first sidewall 121, staggered protrusion structures 128 on two sides of the first sidewall 121 reduce the cross-sectional area of the microneedle, thus decreasing resistance when the microneedle penetrates the skin and further minimizing skin damage and reducing the sensation of pain.

More preferably, at the junction where the third sidewall 123 of the microneedle provided with the protrusion structure 128 is connected with the base layer 11, an easy-to-tear opening is provided. The easy-to-tear opening is a notch formed at the junction of the third sidewall 123 and the base layer 11. When using the microneedle patch 2, after inserting the microneedle 12 into the skin, the base layer 11 can be easily torn off by applying force at the easy-to-tear opening. Additionally, since the microneedle 12 is provided with the protrusion structure 128, there is a greater gripping force between the microneedle 12 and the skin, allowing the microneedle 12 to be stably embedded in the skin. This facilitates the quick separation of the base layer 11 from the microneedle 12, preventing discomfort caused by prolonged adhesion of the base layer 11 to the skin during use of the microneedle patch 2. This design is particularly suitable for children, pets, or psychiatric patients and can prevent the microneedle patch 2 from falling off due to scratching or licking of the base layer 11 by the user.

### Embodiment 3

Building on Embodiment 1, the present embodiment provides a planar microneedle manufacturing device, including a base 31, a mold 32, and a panel 33 positioned above the base 31.

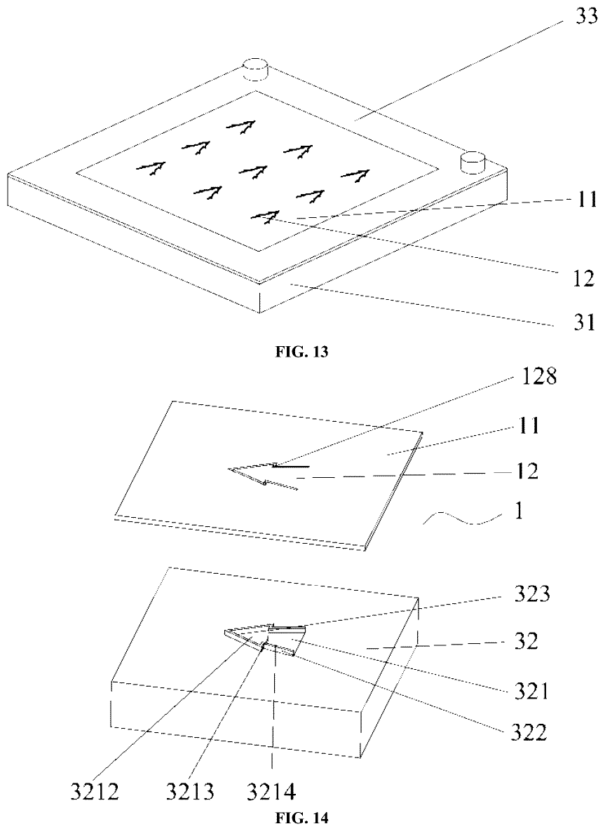

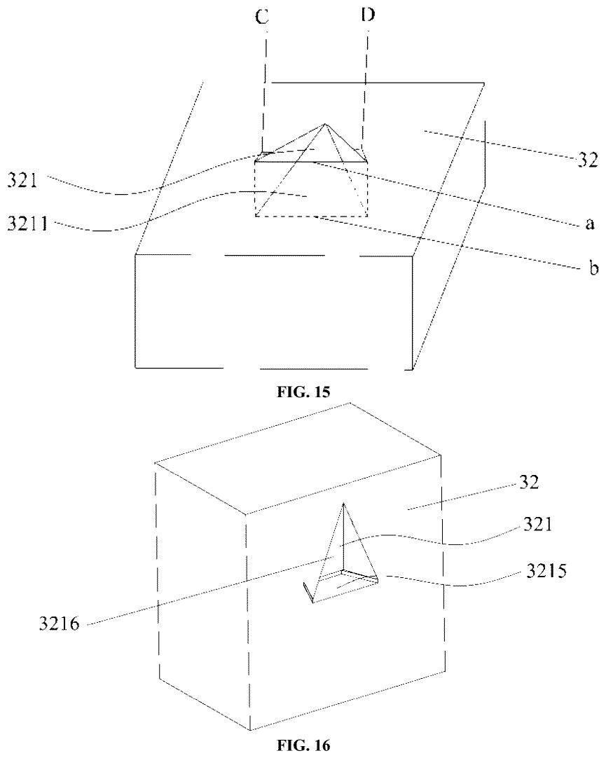

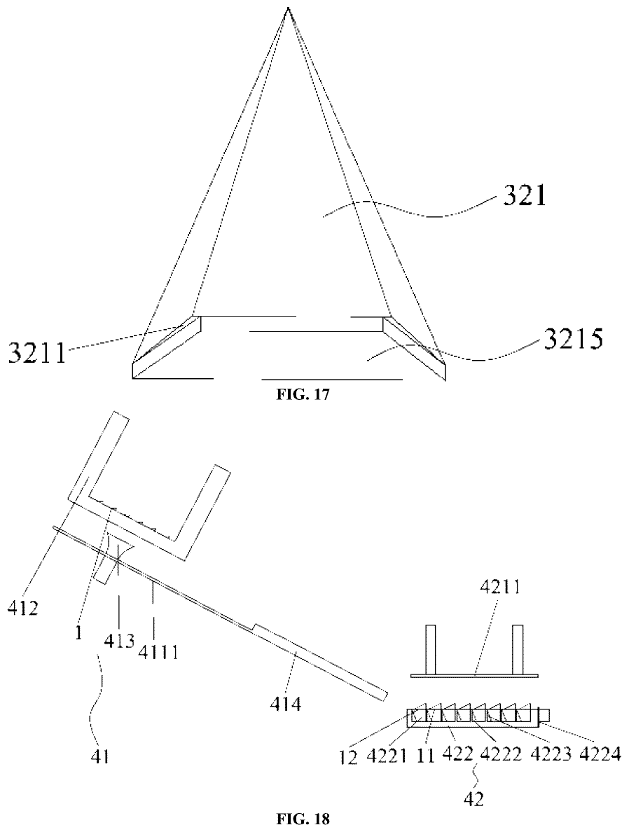

Referring to FIGS. 11-18, the top of the base 31 is provided with a recess 311 to accommodate the mold 32. The top of the mold 32 is provided with multiple forming groove 321 for forming the microneedles 12. Each forming groove 321 includes a needle tip groove 3212, a needle base groove 3214, and a middle part groove 3213, with the middle part groove 3213 communicating with the needle tip groove 3212 and the needle base groove 3214. The direction of the needle tip groove 3212 of the forming groove 321 towards the needle base groove 3214 is a horizontal direction. The panel 33 is provided with a forming hole 331 used for accommodating all the forming grooves 321, and the forming hole 331 serves to form the base layer 11. The top of the mold 32 protrudes upwards to form a separation portion 322, and the separation portion 322 is circumferentially arranged around the forming-groove sidewall 3216 of the forming groove 321. When the mold 32 is embedded into the recess 311 of the base 31, and the panel 33 is pressed against the tops of the base 31 and the mold 32, the height of the separation portion 322 is at least equal to the height of the panel 33.

In the prior art, the microneedle manufacturing device typically uses a raw material solution to form a microneedle patch 2 in one step, with the microneedles 12 extending in a direction perpendicular to the base layer 11, where the ratio of the maximum depth to the maximum length of the forming groove 321 in the mold 32 is generally 2 to 5. However, in the planar microneedle manufacturing device of the present embodiment, the forming groove 321 for forming the microneedle 12 is configured such that the direction of the needle tip groove 3212 of the forming groove 321 towards the needle base groove 3214 is a horizontal direction, where the ratio of the maximum depth to the maximum length of the forming groove 321 is between 1:5 and 1:2.

Compared to the prior art, the forming groove 321 in this embodiment can greatly reduce the depth and the ratio of maximum depth to maximum length of the forming groove 321. Consequently, the planar microneedle manufacturing device in the present embodiment can accurately fill the raw material solution containing medicinal active ingredients into the needle tip groove 3212 of the forming groove 321, which prevents the raw material solution from splashing and contaminating the middle part groove 3213 and the needle base groove 3214. The middle part groove 3213 and the needle base groove 3214 of the forming groove 321 are filled with the raw material solution that does not contain medicinal active ingredients. As a result, the formed microneedle 12 carries the drug only at the needle tip 124, enabling precise control of the drug dosage during the use of the microneedle patch 2. With the ratio of maximum depth to maximum length of the forming groove 321 being between 1:5 and 1:2, the aspect ratio (depth-to-length ratio) of the forming groove 321 is small. Therefore, the raw material solution can be filled without the need for vacuum suction, centrifugation, or high-pressure filling, which shortens the manufacturing process and reduces costs. Meanwhile, because the forming groove 321 is provided in a generally horizontal direction, the demolding of the needle tip 124 and the middle part 125 of the microneedle 12 is not affected by the shape of the middle part 125 and/or the needle base 126. The volume of the needle tip 124 can be greater than that of the middle part 125 and/or the needle base 126. That is, the microneedle 12 may be provided in various shapes, such as an arrow, a tower, a gourd, or a combination of an upper semi-cone and lower semi-frustum, or other structure, or protrusion structures 128 may be formed at the needle tip 124 of the microneedle, so as to enhance the embedding stability of the needle tip 124 after penetrating the skin and preventing the microneedle 12 from being extruded due to skin deformation, ensuring that the medicinal active ingredients in the microneedle 12 are dissolved in the skin, enabling precise drug delivery.

Preferably, the microneedle 12 is in the shape of an arrow, a tower, a gourd, or a combination of an upper semi-cone and lower semi-frustum, or other structure. When the microneedle 12 is in the shape of the arrow, the tower, the gourd, or the combination of an upper semi-cone and lower semi-frustum, or other structure, the needle tip 124 and/or middle part 125 form a protruding portion 127 with larger volume of the microneedle 12, which enhances the gripping force between the microneedle 12 and the skin. After the microneedle 12 penetrates the skin, and the skin deforms due to the pressing and then returns to its original shape, the microneedle 12 remains more stably embedded within the skin, resulting in a stronger gripping force of the microneedle 12 on the skin.

In the present embodiment, by reducing the depth of the forming groove 321, it becomes easier to fill high-viscosity raw material solution, oil-water mixture raw material solution, and other raw material solution into the forming groove 321 to meet the need for forming the microneedle 12 using high-viscosity raw material solution, oil-water mixture raw material solution. and other raw material solution. High-viscosity raw material solution droplets tend to be relatively large in volume. In existing microneedle molds, where the microneedle is formed perpendicular to the base layer, the high aspect ratio of the forming groove causes that once filled into the forming groove, the high-viscosity solution droplets cannot be completely filled into the needle tip portion of the microneedle due to large volume, high viscosity and poor flowability, which hampers scalability of industrialization and results in a very low yield, thereby increasing costs. Alternatively, complete filling and forming of the microneedle might only be achieved under stringent conditions, significantly raising production costs and wasting resources. Oil-water mixture raw material solution easily result in suspensions that increase the viscosity of the raw material solution, leading to the same issues. In the present embodiment, due to the small aspect ratio of the forming groove 321, the entry dimension of the forming groove 321 is also larger, allowing that the high-viscosity raw material solution is rapidly and completely filled into the forming groove 321 by means of scrapping and levelling the raw material solution with the aid of tools, achieving high-yield industrial production and reducing manufacturing costs, thus meeting demand. Additionally, the planar microneedle manufacturing device in the present embodiment is able to reduce the demolding resistance between the microneedle 12 and the forming groove 321, preventing breakage of the microneedle 12, improving the yield of microneedle 12, and lowering costs.

Referring to FIG. 15, preferably, the length of the lower edge b of the forming-groove bottom wall 3211 of the forming groove 321 is less than or equal to the length of the upper edge a. The lower edge b of the forming-groove bottom wall 3211 correspondingly forms the third edge 1233 of the microneedle 12, while the upper edge a correspondingly forms the first edge 1231 of the microneedle 12. When the length of the lower edge b of the forming-groove bottom wall 3211 is larger than that of the upper edge a, the length of the third edge 1233 of the formed microneedle 12 is greater than the length of the first edge 1231. During demolding, the third edge 1233 will contact the forming groove 321, increasing the resistance to demolding of the microneedle 12 and leading to damage to the microneedle 12 or even making the microneedle unable to be demolded. Therefore, in the present embodiment, the length of the lower edge b of the forming-groove bottom wall 3211 is preferably less than or equal to the length of the upper edge a, such that the length of the third edge 1233 of the formed microneedle 12 is less than or equal to the length of the first edge 1231, facilitating the demolding of the microneedle 12 and aiding in the standing of the microneedle 12.

Furthermore, when forming-groove bottom wall 3211 rotates around the upper edge until the forming-groove bottom wall 3211 abuts against the top wall of the mold 32, the length of the lower edge b is greater than the length of the line connecting the two intersection points CD between the lower edge b and the forming groove 321, such that after the formed microneedle 12 stands up, the length of the line connecting the two connection points FG of the third edge 1233 and the second edge 1232 of the third sidewall 123 of the microneedle 12 is greater than the length of the projection edge AB formed by the line being projected onto the base through-hole 112, and further, the third edge 1233 of the microneedle 12 is able to abut against the side of the base layer 11 that is close to the microneedle 12.

Furthermore, along the direction from the needle tip groove 3212 of the forming groove 321 towards the needle base groove 3214, the forming-groove bottom wall 3211 extends outwards to form a protruding portion forming groove 3215. This protruding portion forming groove 3215 is used for forming the protruding portion 127 on the third sidewall 123 of the microneedle 12.

Referring to FIG. 14, further, the top of the mold 32 protrudes upwards to form a separation portion 322, and the separation portion 322 circumferentially surrounds the forming-groove sidewall 3216 of the forming groove 321. When the mold 32 is fitted into the recess 311 of the base 31, and the panel 33 is positioned on tops of the base 31 and the mold 32, the height of the separation portion 322 is at least equal to the height of the panel 33. In the preparation of the planar microneedle 1, the base 31, mold 32, and panel 33 are first assembled, and the raw material solution is filled through the forming hole 331 of the panel 33. The raw material solution enters the forming groove 321 of the mold 32. The raw material solution within the forming hole 331 forms the base layer 11, while the raw material solution within the forming groove 321 forms the microneedle 12. The separation portion 322 located at the top of the mold 32 separates the raw material solution, resulting in a gap formed at the position of the formed base layer 11 corresponding to the separation portion 322, that is, the gap 111 is presented between the junction of the first sidewall 121 and the second sidewall 122 and the base layer 11. Consequently, after the microneedle 12 stands up, the microneedle 12 may rotate around the first edge 1231 and pass through the gap 111, from the state where the first sidewall 121 and the base layer 11 are located in a same plate to the state where the third sidewall 123 and the base layer 11 are substantially located in a same plane or the third sidewall partially abuts against the base layer 11. At this time, the angle between the third sidewall 123 and the base layer 11 is 0° to ±10°, completing the standing of the microneedle 12.

Preferably, along the width direction of the forming groove 321, two sides of the separation portion 322 protrude outwards to form a first protrusion-fabricating recess 323. The raw material solution within the first protrusion-fabricating recess 323 is used to form the protrusion structures 128 on two sides of the first sidewall 121 of the microneedle 12. The forming-groove sidewall 3216 of the forming groove 321 is provided with a second protrusion-fabricating recess, which is utilized for forming the protrusion structures 128 on the circumferential sidewall of the microneedle 12.

### Embodiment 4

Building upon Embodiments 1-3, the present embodiment further provides a method for preparing the planar microneedle. The method specifically includes the following steps:

A1: preparing the raw material solution, which includes a drug-loaded raw material solution and a base raw material solution.

A2: assembling the base 31, mold 32, and panel 33, placing the mold 32 into the recess 311 of the base 31, with the forming hole 331 of the panel 33 positioned above the mold 32 to accommodate all the forming grooves 321 of the mold 32.

A3: filling the needle tip groove 3212 with the drug-loaded raw material solution, where the drug-loaded raw material solution dries to form the needle tip 124 of the microneedle 12.

A4: filling the forming hole 331 with the base raw material solution until the forming hole 331 and the panel 33 are filled up, then scraping and leveling the base raw material solution along the top of the panel 33, where the base raw material solution within the forming hole 331 dries to form the base layer 11, the base raw material solution in the needle base groove 3214 dries to form the needle base 126, and the base raw material solution in the middle part groove 3213 dries to form the middle part 125.

The separation portion 322 located at the top of the mold 32 separates the base raw material solution, resulting in a gap formed at the position of the formed base layer 11 corresponding to the separation portion 322. That is, a gap 111 is formed between the junction of the first sidewall 121 and the second sidewall 122, and the base layer 11.

A5: removing the panel 33 to separate the microneedle 12 from the forming groove 321.

Specifically, step A1 further includes:
when the drug is a water-soluble drug, the drug-loaded raw material solution contains a sodium hyaluronate solution, active ingredients, and water. The sodium hyaluronate solution contains 5% to 50% sodium hyaluronate solution with a molecular weight of 30,000 to 300,000, as well as 1% to 20% sodium hyaluronate solution with a molecular weight smaller than or equal to 10,000. The proportion of active ingredients is 1% to 20% the drug-loaded solution. The proportion of water is 50% to 80% the drug-loaded solution. The solid content of the drug-loaded solution is 20% to 60%.

When the drug is an oil-soluble drug, the drug-loaded raw material solution contains a sodium hyaluronate solution and an oil-soluble drug active ingredient solution. The density of the oil-soluble drug active ingredient solution is greater than 1 and is close to the density of the sodium hyaluronate solution. Due to that the close densities between the oil-soluble drug active ingredient solution and the sodium hyaluronate solution, the oil-soluble drug active ingredients may be stably suspended within the sodium hyaluronate solution, and do not sink or float during drying of the drug-loaded solution, ensuring that the oil-soluble drug active ingredients are stably distributed within the sodium hyaluronate after drying of the drug-loaded solution. The sodium hyaluronate may be replaced with other soluble microneedle scaffold materials.

When the microneedle 12 is insoluble in water and human tissue fluid for the purpose of tissue fluid extraction and detection, the drug-loaded raw material solution contains a component solution and a 2% calcium chloride ethanol solution. In the above, the component solution contains: 15% sodium alginate. 10% polyvinyl alcohol, and 75% water. For use, the three components in the component solution are mixed evenly and then heated to dissolve at a temperature of 60 to 65° C. followed by heat preservation and degassing for later use. The 2% calcium chloride ethanol solution contains 2% calcium chloride and 98% anhydrous ethanol. For use, the two components in the 2% calcium chloride ethanol solution are stirred and dissolved for later use. After the planar microneedle 1 is formed, the planar microneedle 1 is immersed in the 2% calcium chloride ethanol solution for 1 to 2 minutes, allowing a chemical reaction to occur between the sodium alginate and calcium chloride to produce calcium alginate. Calcium alginate is insoluble in water and human tissue fluid, making it suitable for tissue fluid extraction and detection. Subsequently, the microneedle is dried in a forced air drying oven at 35° C. for 20 to 30 minutes.

Specifically, step A4 further includes:
scrapping and leveling the drug-loaded solution in the direction of the needle tip groove 3212 of the mold 32 towards the needle base groove 1214, where since the depth thereof increases gradually in the direction of the needle tip groove 3212 towards the needle base groove 1214, when scrapping and leveling the drug-loaded solution in this direction, the gas within the forming groove 321 is expelled in the direction of the needle tip groove 3212 towards the needle base groove 1214 as the raw material solution fills the forming groove 321, preventing the presence of air bubbles in the formed microneedle 12 and ensuring that the microneedle 12 has precise drug loading and a strength meeting the requirements.

Alternatively, the raw material solution may be filled into the forming hole 331 of the panel 33 until the forming hole 331 is filled up, the raw material solution is scrapped and leveled along the top of the panel 33, and then it is placed in a vacuum chamber and subjected to vacuuming for 2 minutes under a vacuum environment of −0.2 Mpa to remove the gas within the raw material solution in the forming groove 321, preventing the presence of air bubbles in the formed microneedle 12. This vacuuming process requires low power and a short time. helping to conserve energy. reduce the forming time of microneedle 12, and improve production efficiency.

Alternatively, the raw material solution may be filled into the forming groove 331 of the panel 33 until the forming hole 331 is filled up, the raw material solution is scrapped and leveled along the top of the panel 33, and then it is placed in a centrifuge and centrifuged at a rotation speed of 200 r/min for 1 minute. followed by centrifuging at 1.000 r/min for 3 minutes to expel the gas within the raw material solution in the forming groove 321, thereby avoiding the presence of air bubbles in the formed microneedle 12. This centrifugation process requires low power and a short duration, resulting in energy savings, reduced forming time for microneedle 12, and enhanced production efficiency.

Specifically, step A4 further includes:

During the drying process:
after filling the raw material solution into the forming hole 331 of the panel 33, it is dried in an environment with 25% to 30% relative humidity for 20 to 30 minutes to achieve the first stage of rapid drying. Subsequently, it is dried in an environment with 40% to 60% relative humidity for 10 to 30 minutes to achieve the second stage of slow drying. This combined drying method of rapid drying and slow drying helps to prevent the planar microneedle 7 from curling or deforming during the drying process, which in turn ensures the quality of the finished planar microneedle 1, reduces the drying time of the planar microneedle 1, shortens the production cycle of the planar microneedle 1, enhances production efficiency of the planar microneedle 1, and lowers production costs.

Alternatively, following the filling of the raw material solution into the forming hole 331 of the panel 33, the solution may be subjected to slow drying overnight within a drying oven maintained at a relative humidity of 25%-60%, to ensure that the planar microneedle 1 remains free from curling or deformation during the drying, thereby safeguarding the quality of the finished planar microneedle 1.

Specifically, step A5 further includes:
after the raw material solution is dried, removing the panel 33, and applying an upward suction force to the base layer 11 using vacuum suction to quickly separate the base layer 11 from the mold 32, facilitating the separation of the microneedle 12 from the forming groove 321 of the mold 32, thereby forming the planar microneedle 1.

Alternatively, after the raw material solution is dried, the panel 33 is removed, and the base layer 11 is lifted in the direction from the needle base 126 of the microneedle 12 towards the needle tip 124 to separate the microneedle 12 from the forming groove 321, thereby forming the microneedle patch 2. In this process, the base layer 11 is lifted in the direction from the needle base 126 towards the needle tip 124 of the microneedle 12, applying force at the junctions of the first sidewall 121 and the third sidewall 123 of the microneedle 12 with the base layer 11 to drive the microneedle 12 separated from the forming groove 321, avoiding that fractures at the junctions of the first sidewall 121 and the third sidewall 123 of the microneedle 12 with the base layer 11 lead to separation of the microneedle 12 and the base layer 11 and then defective products, and improving the yield of the planar microneedle 1 and reducing production costs of the planar microneedle 1.

Above all, the aforementioned preparation method is simple to operate and highly efficient. In steps A3 and A4, the drug-loaded raw material solution and the base raw material solution may be filled using methods such as high-pressure filling, vacuum suction, or centrifugation.

### Embodiment 5

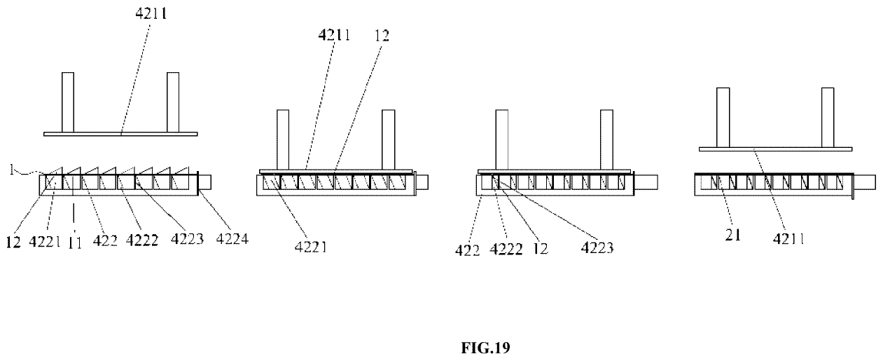

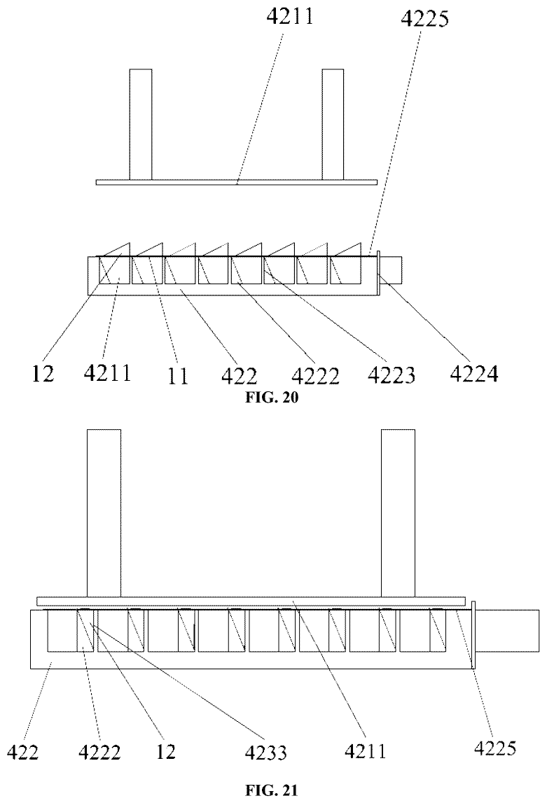

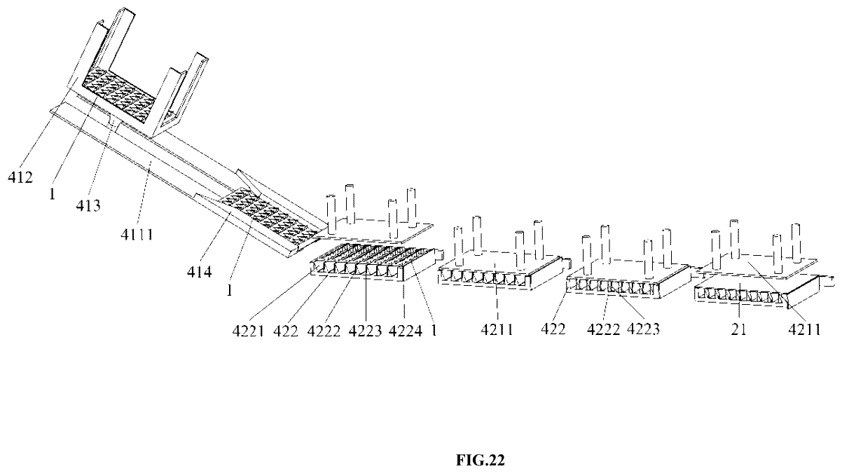

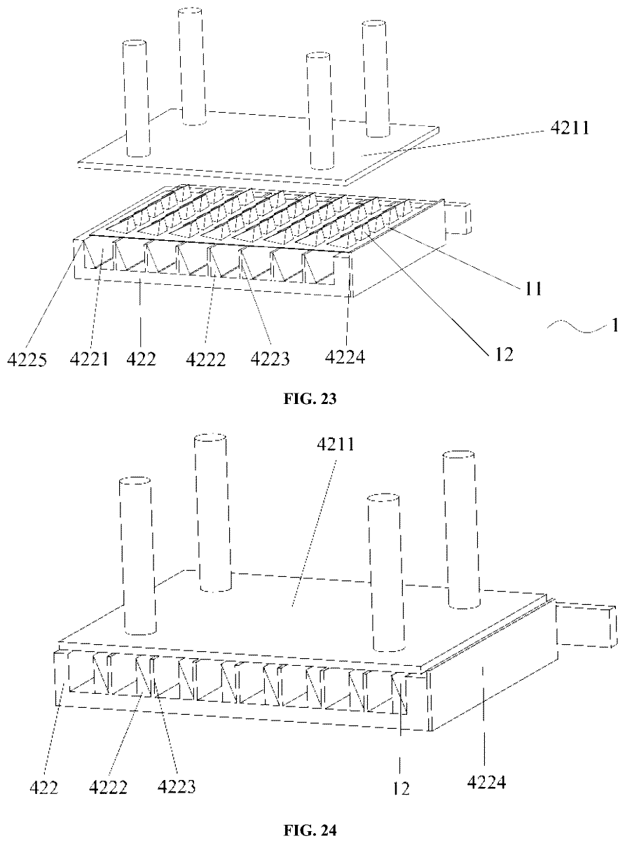

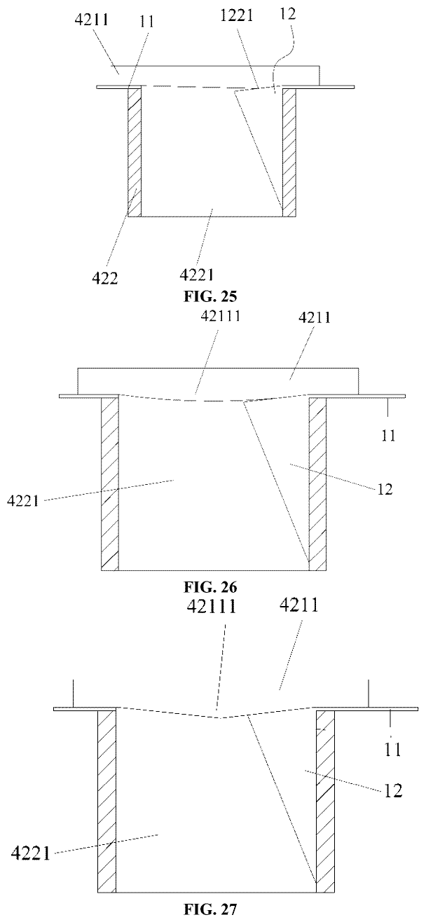

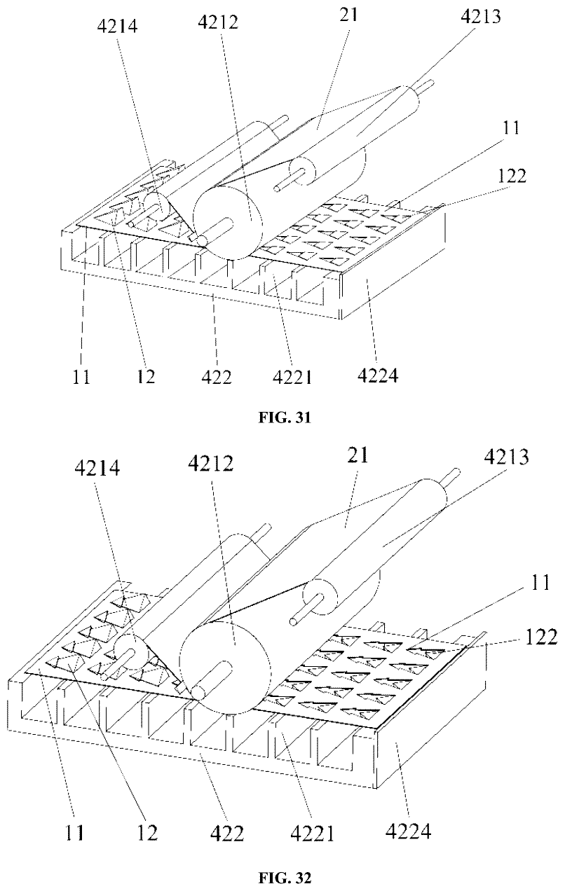

Referring to FIGS. 18-33, based on Embodiments 1-2, the present embodiment further provides a planar microneedle standing device, used to apply pressure to the microneedle 12 of the planar microneedle 1 to cause the microneedle 12 to rotate around the first edge 1231 and pass through the gap 111, such that the microneedle 12 rotates from the state where the first sidewall 121 of the microneedle 12 and the base layer 11 are located in the same plane to the state where the third sidewall 123 and the base layer 11 are approximately in the same plane or partially abut against the base layer 11. At this time, the angle between the third sidewall 123 and the plane where the base layer 11 is located is 0° to ±10°, thereby completing the standing of the microneedle 12. At this point, a base through-hole 112 is formed at the junction between the microneedle 12 and the base layer 11.

Furthermore, the planar microneedle standing device includes a planar microneedle conveying assembly 41 and a planar microneedle rotating assembly 42. The planar microneedle conveying assembly 41 is used to transport the planar microneedle 1 to the planar microneedle rotating assembly 42, and the planar microneedle rotating assembly 42 is used for making the microneedle 12 stand up.

Furthermore, the planar microneedle conveying assembly 41 includes a planar microneedle conveyor belt 4111, a storage device 412, and a planar microneedle adsorber 413. The storage device 412 is used to store planar microneedles 1, with the multiple planar microneedles 1 stacked up and down. The planar microneedle conveyor belt 4111 is positioned beneath the storage device 412, and the planar microneedle adsorber 413 is arranged on the planar microneedle conveyor belt 4111. The planar microneedle adsorber 413 is used for adsorbing the planar microneedles 1 one by one onto the planar microneedle conveyor belt 4111, and the planar microneedle conveyor belt 4111 is used for transporting the planar microneedles 1 to the planar microneedle rotating assembly 42.

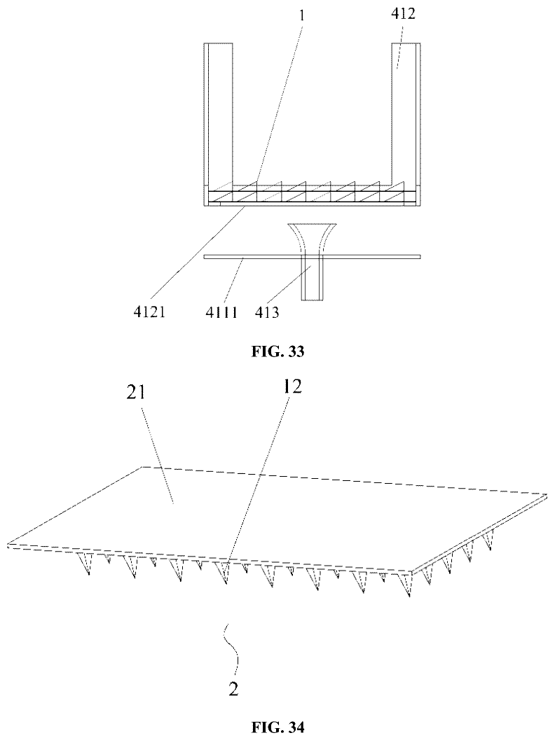

Referring to FIG. 33, specifically, the bottom of the storage device 412 is provided with a discharge port 4121 for discharging the planar microneedles 1. The discharge port 4121 is specifically embodied as: having a shape similar to that of the planar microneedle 1 and an area smaller than that of the planar microneedle 1, for storage of the planar microneedle 1. The discharge port 4121 is arranged parallel to the planar microneedle conveyor belt 4111, with a spacing between the discharge port 4121 and the planar microneedle conveyor belt 4111 being greater than the thickness of the planar microneedle 1.

The planar microneedle adsorber 413 is provided extending through the planar microneedle conveyor belt 4111. The planar microneedle adsorber 413 can move axially and is capable of discharging a pulsed negative pressure airflow to sequentially adsorb the planar microneedles 1 from the discharge port 4121 and lay them flat onto the planar microneedle conveyor belt 4111. When adsorption of the planar microneedles 1 is required, the planar microneedle adsorber 413 moves upwards along its axial direction and approaches the bottom of the storage device 412, and discharges a pulsed negative pressure airflow. Once the adsorption is complete, the planar microneedle adsorber 413 moves downwards along its axial direction, returns to a position beneath the planar microneedle conveyor belt 4111 to transfer the planar microneedles 1 onto the planar microneedle conveyor belt 4111.

Preferably, the planar microneedle adsorber 413 is positioned beneath the center of the planar microneedles 1 within the storage device 412, allowing the adsorption force exerted by the planar microneedle adsorber 413 to act on the center of the planar microneedle 1. When the planar microneedle 1 deforms and is released from the discharge port 4121, the center of the planar microneedle 1 first adheres onto the planar microneedle adsorber 413. Subsequently, this adsorption force creates a tensile force at the edge of the planar microneedle 1. Under the influence of this tensile force, the edge of the planar microneedle 1 extends and lays flat on the planar microneedle conveyor belt 4111, thereby facilitating the movement and lay flat of the planar microneedles 1 from the storage device 412 onto the planar microneedle conveyor belt 4111.

Specifically, both sides of the planar microneedle conveyor belt 4111 are provided with blocking plates 414, where the blocking plates 414 serve to guide the movement of the planar microneedles 1 when the planar microneedles 1 are conveyed by the planar microneedle conveyor belt 4111.

Further, the planar microneedle rotating assembly 42 includes a rotating mechanism and a microneedle accommodating mechanism 422. The rotating mechanism is used for rotating the microneedle 12 from a state where the first sidewall 121 of the microneedle 12 and the base layer 11 are located in the same plane to a state where the third sidewall 123 is approximately in the same plane as the base layer 11 or partially abuts against the base layer 11, thereby completing the standing of the microneedle 12. The top of the microneedle accommodating mechanism 422 is provided with an accommodating groove 4221, where the standing/upright microneedle 12 is accommodated in the accommodating groove 4221. The top wall 4225 of the top of the microneedle accommodating mechanism 422 may support the base layer 11, while the sidewalls of the accommodating groove 4221 provide support to the microneedle 12, avoiding damage to the base layer 11 and the microneedle 12 during the standing process of the microneedle 12, when the rotating mechanism applies pressure to the planar microneedle 1.

The rotating mechanism is provided on a frame (not shown in the drawings), while the microneedle accommodating mechanism 422 is provided on a standing conveyor belt 4112 (also not shown in FIG. 33). The standing conveyor belt 4112 is used to drive the horizontal movement of the microneedle accommodating mechanism 422.

The length of the accommodating groove 4221 is greater than the height of the microneedle 12 from the needle tip 124 to the needle base 126, thereby avoiding the microneedle 12 from colliding with the accommodating groove 4221 during the standing process to damage it. thus ensuring a higher yield of the microneedle 12 after standing. The depth of the accommodating groove 4221 is greater than the height of the microneedle 12 from the needle tip 124 to the needle base 126 to prevent the microneedle 12 from colliding with the bottom of the accommodating groove 4221 to make it damaged, when it is housed within the accommodating groove 4221 after standing, further ensuring a higher yield of the microneedle 12 after standing. As both the length and depth of the accommodating groove 4221 are greater than the height of the microneedle 12 from the needle tip 124 to the needle base 126, this facilitates the separation of the microneedle 12 from the accommodating groove 4221 when the planar microneedle 1 is transformed into the microneedle patch 2.

Specifically, multiple accommodating grooves 4221 are provided, with multiple accommodating grooves 4221 arranged at intervals on the top of the microneedle accommodating mechanism 422. The multiple accommodating groove 4221 corresponds one-to-one with the multiple rows of microneedles 12 on the planar microneedle 1.

Further, the microneedle accommodating mechanism 422 is preferably made from an elastic material, such as silicone or rubber, allowing the top wall 4225 of the top of the microneedle accommodating mechanism 422 to provide elastic support for the base layer 11. The sidewalls of the accommodating groove 4221 may offer elastic support for the microneedles 12. When the rotating mechanism applies pressure to the planar microneedle 1, both the top wall 4225 of the microneedle accommodating mechanism 422 and the base layer 11 are subjected to compression simultaneously. The top wall 4225 may provide a reactive force to support the base layer 11, and the top wall 4225 may undergo elastic deformation along with the base layer 11 in response to compression, thereby preventing damage to the base layer 11. The sidewalls of the accommodating groove 4221 may provide a reactive force to support the microneedles 12, and the sidewalls of the accommodating groove 4221 are capable of elastically deforming along with the microneedle 12 when subjected to pressure, preventing damage to the microneedle 12.

Further, one end of the microneedle accommodating mechanism 422 away from the planar microneedle conveyor belt 4111 is provided with a limiting plate 4224. The limiting plate 4224 is capable of vertically moving. When the planar microneedle 1 is positioned at the top of the microneedle accommodating mechanism 422, the planar microneedle 1 abuts against the limiting plate 4224, thereby enhancing the positional accuracy of the planar microneedle 1.

Further, the rotating mechanism includes a pressing plate 4211, or the rotating mechanism includes a first roller 4212 or the rotating mechanism includes a roller assembly.

Referring to FIGS. 18-27, further, when the rotating mechanism includes the pressing plate 4211:
the pressing plate 4211 is made of an elastic material, the pressing plate 4211 may rotate all the microneedles 12 of the planar microneedle 1 at the same time to make them stand up. thereby enhancing the efficiency of the standing.

The pressing plate 4211 is installed to a frame (not shown). The pressing plate 4211 has a uniform thickness in the vertical direction. The pressing plate 4211 may move downwards relative to the frame in the vertical direction to make contact with the third sidewall 123 of the microneedle 12 and apply pressure against the third sidewall 123, so as to cause the microneedle 12 to rotate around the first edge 1231 until the third sidewall 123 is approximately in the same plane as the base layer 11, or partially abuts against the base layer 11. That is, the angle between the third sidewall 123 and the plane where the base layer 11 is located is 0° to ±10°, thereby completing the standing of the planar microneedle 1.

Alternatively, the pressing plate 4211 is installed to a frame (not shown), with one side of the pressing plate 4211 distant from the planar microneedle 1 being connected to the frame via a spring or other elastic structure (not shown). The pressing plate 4211 is capable of moving in the vertical direction relative to the frame. The pressing plate 4211 is in the form of a wedge, and the pressing plate 4211 has a triangular or trapezoidal cross-section in the vertical direction. The extending direction of the wedge-shaped pressing plate 4211 aligns with the direction of the needle base 126 of the microneedle 12 of the planar microneedle 1 towards the needle tip 124. That is, when the third sidewall 123 of the microneedle 12 is provided away from the planar microneedle conveyor belt 4111, the thickness of the end of the pressing plate 4211 that is remote from the planar microneedle conveyor belt 4111 is greater than the thickness of the end thereof that is closer to the planar microneedle conveyor belt 4111. When the pressing plate 4211 moves downwards to press against the planar microneedle 1, the microneedle 12, under the action of the wedge-shaped pressing plate 4211, establishes contact between the third sidewall 123 of the microneedle 12 and the inclined surface of the wedge-shaped pressing plate 4211, forming a contact point. The pressing plate 4211 exerts a component force on the third sidewall 123 that is directed from the contact point towards the needle tip 124, thereby causing the microneedle 12 to rotate around the first edge 1231. As the pressing plate 4211 continues to press downwards, the third sidewall 123 is subjected to a component force from the pressing plate 4211 in the horizontal direction, as well as a component force acting vertically downwards, so as to make the microneedle 12 continue to rotate around the first edge 1231 until the third sidewall 123 is approximately in the same plane as the base layer 11, or partially abuts against the base layer 11. That is, the angle between the third sidewall 123 and the plane where the base layer 11 is located is 0°˜±10°, thereby completing the standing of the planar microneedle 1. Since the side of the pressing plate 4211 distant from the planar microneedle 1 is connected to the frame via a spring or other elastic structure, when the wedge-shaped pressing plate 4211 presses down on the planar microneedle 1, the pressure between the pressing plate 4211 and the planar microneedle 1 may be adjusted through the spring structure. so as to prevent excessive pressure on the planar microneedle 1 which could lead to deformation and thus defective products.

Alternatively, the pressing plate 4211 is installed on a frame (not shown), and the pressing plate 4211 has a uniform thickness in the vertical direction, and is capable of moving in an inclined downward direction relative to the frame. That is, the pressing plate 4211 moves downwards inclinedly relative to the planar microneedle 1 in the direction from the contact point between the third sidewall 123 and the pressing plate 4211 towards the needle tip 124. In relation to the horizontal direction, the inclined angle is 30°≤α≤60°. The pressing plate 4211 moves inclinedly downwards in a direction from the contact point between the third sidewall 123 and the pressing plate 4211 towards the needle tip 124. When the pressing plate 4211 makes contact with the third sidewall 123, forming the contact point, and applies pressure to the third sidewall 123, it exerts on the third sidewall 123 a component force directed from the contact point towards the needle tip 124, so as to cause the microneedle 12 to rotate around the first edge 1231. As the pressing plate 4211 continues to press downwards, the third sidewall 123 experiences a horizontal component force and a vertical downward component force from the pressing plate 4211, to make the microneedle 12 continue to rotate around the first edge 1231 until the third sidewall 123 is approximately in the same plane as the base layer, or partially abuts against the base layer 11. That is, the angle between the third sidewall 123 and the plane where the base layer 11 is located is 0° to ±10°, thereby completing the standing of the planar microneedle 1. When α<30°, the pressing plate 4211 needs to move a greater distance downwards to make contact with the microneedle 12. If the area of the pressing plate 4211 is substantially the same as that of the planar microneedle 1, then when the pressing plate 4211 is fully pressed against the surface of the base layer 11 of the planar microneedle 1, some microneedles 12 may be outside the boundaries of the pressing plate 4211. In this case, the pressing plate 4211 cannot make contact with those microneedles 12 to apply pressure thereto, making it impossible to achieve complete standing. If the area of the pressing plate 4211 is significantly larger than that of the planar microneedle 1, the standing of all microneedles 12 on the planar microneedle 1 can be achieved, but the number of planar microneedles 1 that can be placed on a production line of the same length may relatively decrease, thereby reducing production efficiency and indirectly increasing production costs. When α>60°, after the pressing plate 4211 is fully pressed against the surface of the base layer 11, the portion of the base layer 11 that comes into contact with the pressing plate 4211 first is subjected to greater stress, which can easily lead to deformation, resulting in defective products and indirectly increasing production costs.

Referencing FIGS. 26-27, it is preferred that the bottom of the pressing plate 4211 is provided with multiple pressure application portions 42111, where the pressure application portions 42111 correspond one-to-one with the accommodating groove 4221. The longitudinal cross-section of the pressure application portion 42111 may be in the shape of a downwardly protruding arc, V-shape, or rectangle and other configurations capable of applying pressure to the microneedle 12. It is preferred that the longitudinal cross-section of the pressure application portions 42111 is in the shape of a downwardly protruding arc or V-shape, such that the shape of the pressure application portions 42111 matches the shape of the deformed third sidewall 123 of the microneedle 12 when the pressure application portions 42111 applies pressure to the microneedle 12, thereby ensuring that the third sidewall 123 of the microneedle 12 experiences uniform stress and preventing damage to the microneedle 12.

When the microneedle 12 does not have a protruding portion 127, it is preferred that both the material of the pressing plate 4211 and the material of the pressure application portions 42111 are elastic materials, such as rubber or silicone, such that when the pressing plate 4211 or the pressure application portion 42111 applies pressure to the microneedle 12, the pressing plate 4211 or the pressure application portion 42111 may deform and sink into the accommodating groove 4221, thereby providing sufficient rotating force for the microneedle 12 to rotate the microneedle 12 from the state where the first sidewall 121 is positioned in the same plane as the base layer 11 to the state where the third sidewall 123 is inclined towards the side of the base layer 11 that is closer to the microneedle 12. Subsequently, upon the rebound of the microneedle 12, the third sidewall 123 of the microneedle 12 can be located approximately in the same plane as the base layer 11, or partially abut against the base layer 11, such that the angle between the third sidewall 123 and the plane where the base layer 11 is located is 0° to ±10°, thereby completing the standing of the microneedle 12.

When the microneedle 12 is provided with a protruding portion 127, the required rotating force is relatively small since the pressing plate 4211 or the pressure application portion 42111 only needs to rotate the microneedle 12 to make the protruding portion 127 flush with the side of the base layer 11 that is away from the microneedle 12. Therefore, the material of the pressing plate 4211 or the pressure application portions 42111 is not limited. It may be a rigid material or elastic material, reducing the difficulty in selecting materials for the pressing plate 4211 and the pressure application portions 42111.

Further, after the pressing plate 4211 has completed the standing of the microneedle 12 and the microneedle patch 2 with an adhesion layer 21 needs to be prepared, it is preferred that the microneedle accommodating mechanism 422 also includes multiple fixing plate assemblies arranged at intervals. The fixing plate assembly is used to prevent, when the pressing plate 4211 returns to the original position and moves away to no longer press the planar microneedle 1, the microneedle 12 from rotating around the first edge 1231 without external force in the direction opposite to that during the standing, which rotation results in a certain angle between the third sidewall 123 and the base layer 11, leading to potential trapping of gas between the third sidewall 123 and the adhesion layer 21 during the adhesion of the adhesion layer 21 to create wrinkles and causes defective products.

Each fixing plate assembly includes a first clamping plate 4222 and a second clamping plate 4223. The first clamping plate 4222 is capable of moving towards or away from the second clamping plate 4223 along a direction parallel to the base layer 11. When the first clamping plate 4222 rests against the second clamping plate 4223 of the same fixing plate assembly, an accommodating groove 4221 is formed between the first clamping plate 4222 and the second clamping plate 4223. After the microneedle 12 stands up and when the microneedle 12 is located within the accommodating groove 4221, the first clamping plate 4222 and the second clamping plate 4223 serve to accommodate and clamp the microneedle 12. For clarity in the following description, the example will consider the scenario where the first clamping plate 4222 is located to the left of the second clamping plate 4223.

Referring to FIGS. 18-24, the specific working principle of the fixing plate assembly is as follows.

In the initial state, the first clamping plate 4222 is positioned away from the second clamping plate 4223 of the same fixing plate assembly, thereby forming an accommodating groove 4221 between the first clamping plate 4222 and the second clamping plate 4223. At this time, the space within the accommodating groove 4221 is relatively large, allowing the microneedle 12 to rotate freely within the accommodating groove 4221 without being obstructed by the first clamping plate 4222. The planar microneedle conveyor belt 4111 transports the planar microneedle 1 to the top of the microneedle accommodating mechanism 422, and makes multiple rows of microneedles 12 on the planar microneedle 1 provided in one-to-one correspondence with the multiple accommodating grooves 4221 at the top of the microneedle accommodating mechanism 422.

The pressing plate 4211 applies pressure to all microneedles 12 on the planar microneedle 1. The microneedle 12 is rotated from the state where the first sidewall 121 of the microneedle 12 is in the same plane as the base layer 11 to the state where the third sidewall 123 of the microneedle 12 is approximately in the same plane as the base layer 11, or partially abuts against the base layer 11. That is, the angle between the third sidewall 123 and the plane where the base layer 11 is located is 0° to ±10°. At this point, the microneedle 12 is positioned within the accommodating groove 4221. Subsequently, multiple first clamping plates 4222 move synchronously to the right. thereby forming accommodating grooves 4221 each having a space between the first clamping plate and the second clamping plate 4223 that is equal to or slightly larger than the space occupied by the microneedle 12, such that the microneedle 12 is located within the accommodating groove 4221 and is constrained by the first clamping plate 4222, preventing rotation of the microneedle 12 around the first edge 1231 in the direction opposite to that during standing. Upon the pressing plate 4211 returns to the original position to leave the pressing position on the planar microneedle 1, the microneedle 12 may still maintain the configuration where the third sidewall 123 is approximately in a same plane as the base layer 11 or partially abuts against the base layer 11. That is, the angle between the third sidewall 123 and the plane where the base layer 11 is located is 0° to ±10°, preventing poor adhesion or wrinkling of the adhesion layer 21 caused by displacement of the microneedle 12 during adhesion of the adhesion layer 21.

Specifically, the microneedle accommodating mechanism 422 is provided with a driving structure, which is utilized to drive the movement of the first clamping plate 4222.

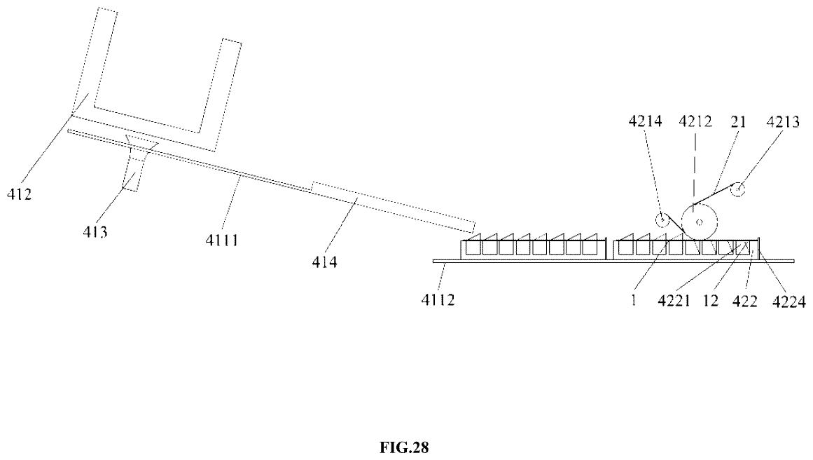

Referring to FIGS. 28-32, further, when the rotating mechanism includes a first roller 4212, the first roller 4212 is pressed against the planar microneedle 1. The first roller 4212 is employed to apply pressure to the microneedle 12, thereby make the microneedle 12 rotate from the state where the first sidewall 121 of the microneedle 12 and the base layer 11 are located in the same plane to the state where the third sidewall 123 is approximately in the same plane as the base layer 11, or partially abuts against the base layer 11. That is, the angle between the third sidewall 123 and the plane where the base layer 11 is located is 0° to ±10°, thereby completing the standing of the microneedle 12. At this moment:
the first roller 4212 and the microneedle accommodating mechanism 422 are capable of moving towards each other, specifically embodied as: the first roller 4212 rotates around the fixed shaft of the first roller 4212, and the microneedle accommodating mechanism 422 moves in the direction away from the planar microneedle conveyor belt 4111 along with a standing conveying belt 4112 (not shown); alternatively, the microneedle accommodating mechanism 422 remains in a fixed position, and the first roller 4212 rolls about the supporting shaft of the first roller 4212 relative to the micro-needle accommodating mechanism 422; alternatively, the microneedle accommodating mechanism 422 moves along with the standing conveyor belt 4112 (not shown) in the direction away from the planar microneedle conveyor belt 4111, and the first roller 4212 rolls around the supporting axis of the first roller 4212 and moves horizontally towards the planar microneedle conveyor belt 4111.

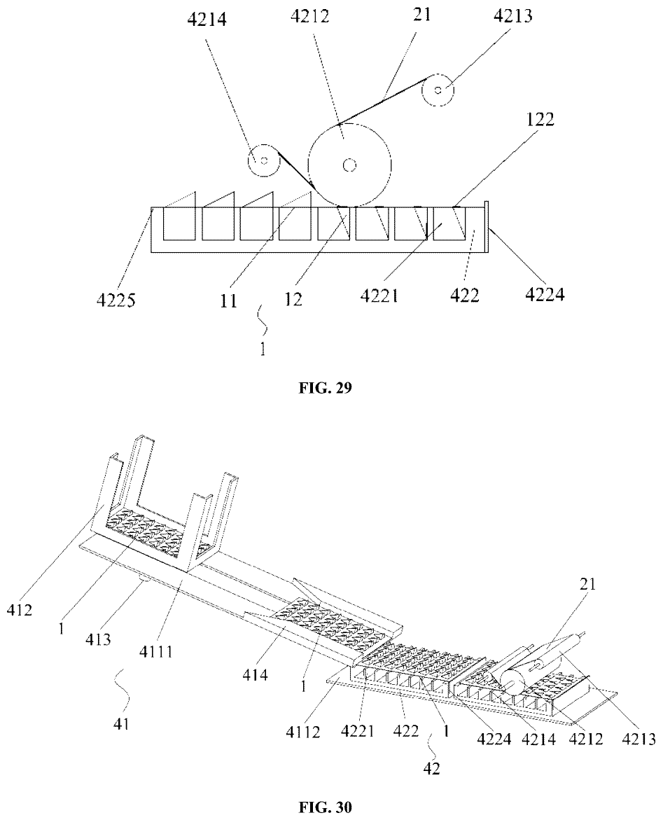

Preferably, the first roller 4212 rolls around the fixed shaft of the first roller 4212, while the microneedle accommodating mechanism 422 moves horizontally away from the planar microneedle conveyor belt 4111 along with the standing conveyor belt 4112 (not shown). This reduces the relative moving elements, simplifies the control elements of the equipment, and streamlines the process. Referring to FIG. 29, the first roller 4212 is positioned on one side opposite to the third sidewall 123 of the microneedle 12 on the planar microneedle 1. The microneedle accommodating mechanism 422 moves in the direction away from the planar microneedle conveyor belt 4111 along with the standing conveyor belt 4112 (not shown). The first roller 4212 rotates in a counterclockwise direction, and the surface of the first roller 4212 presses against the third sidewall 123, applying to the third sidewall 123 a horizontal component force directed towards the planar microneedle conveyor belt 4111, as well as a vertical downward component force, to make the microneedle 12 rotate around the first edge 1231 until the third sidewall 123 is approximately in the same plane as the base layer 11, or partially abuts against the base layer 11. That is, the angle between the third sidewall 123 and the plane where the base layer 11 is located is 0° to ±10°. This completes the standing of the planar microneedle 1. The first roller 4212 applies pressure to the microneedles row by row, thereby facilitating the standing of the microneedles 12 row by row. Because the direction of rotation of the first roller 4212 is the same as the direction of rotation of the microneedle 12, the pressing force applied by the first roller 4212 on the third sidewall 123 of the microneedle 12 is converted into a rotational force that facilitates the rotation of the microneedle 12, preventing damage to the microneedle 12 due to the applied force during the standing process, thereby ensuring the yield of the product.

Further, after the first roller 4212 completes the standing of the microneedle 12 and the microneedle patch 2 with an adhesion layer 21 needs to be prepared, the rotating mechanism further includes a roller assembly. The roller assembly is provided on a frame (not shown) and the roller assembly includes a second roller 4213 and a third roller 4214.

The second roller 4213 has an adhesion layer 21 wound around it, and the second roller 4213 is used to transfer the adhesion layer 21 to the first roller 4212. The side of the adhesion layer 21 that is provided with a protective film faces away from the rotational shafts of the first roller 4212 and the second roller 4213. The first roller 4212 is provided with a protective film peeling structure on the side thereof close to the third roller 4214. This protective film peeling structure is used to control the peeling position of the protective film being peeled off from the adhesion layer 21. The third roller 4214 is used to be wrapped with and store the protective film torn from the adhesion layer 21 on the first roller 4212. This protective film serves to prevent the adhesion layer 21 from adhering to itself during winding to make it unusable. After the protective film of the adhesion layer 21 is removed, the adhesive side faces the base layer 11 of the planar microneedle 1. The first roller 4212 is used to apply pressure to the microneedle 12, facilitating the rotation of the microneedle 12 from the state where the first sidewall 121 of the microneedle 12 and the base layer 11 are located in the same plane to the state where the third sidewall 123 is approximately in the same plane as the base layer 11, or partially abuts against the base layer 11. That is, the angle between the third sidewall 123 and the plane where the base layer 11 is located is 0° to ±10°, thereby completing the standing of the microneedle 12. Simultaneously, the first roller 4212 can also adhere the adhesion layer 21 to the side of the base layer 11 that is away from the microneedle 12, as well as to the third sidewall 123 of the microneedle 12 or the protruding portion 127, to prepare the microneedle patch 2 with the adhesion layer 21.

At this point, the microneedle accommodating mechanism 422 is able to drive the planar microneedle 1 to move in the direction away from the planar microneedle conveyor belt 4111.

The first roller 4212 and the microneedle accommodating mechanism 422 can move towards each other, enabling the standing of the microneedles 12 row by row. The adhesion layer 21 is adhered row by row to the side of the base layer 11 that is away from the microneedle 12, as well as to the third sidewall 123 of the microneedle 12 or the protruding portion 127, thereby forming the microneedle patch 2 with the adhesion layer 21. During this process, the adhesion layer 21 is able to adhere row by row to the base layer 11 and the third sidewall 123 of the microneedle 12 or the protruding portion 127, so as to completely discharge air between the base layer 11 and the adhesion layer 21, and between the third sidewall 123 of the microneedle 12 or the protruding portion 127 and the adhesion layer 21, thereby preventing the formation of wrinkles in the finished microneedle patch 2. At the same time, after the third sidewall 123 or the protruding portion 127 of the microneedle 12 is adhered to the adhesion layer 21, the adhesive force between the base layer 11 and the adhesion layer 21 exerts a pulling effect, ensuring that the portion of the adhesion layer 21 in contact with the third sidewall 123 or the protruding portion 127 of the microneedle 12 remains smooth and wrinkle-free, improving the yield of quality products in the finished microneedle patch 2.

Further, the first roller 4212 is preferably made of an elastic material, such as rubber or silicone, so that when the first roller 4212 applies pressure to the microneedle 12, the first roller 4212 may deform and sink into the accommodating groove 4221, providing sufficient rotating pressure for the microneedle 12 to make the microneedle 12 rotate from the state where the first sidewall 121 of the microneedle 12 is in the same plane as the base layer 11 to the state where the third sidewall 123 of the microneedle 12 is inclined towards the side of the base layer 11 that is closer to the microneedle 12. Subsequently, upon the rebound of the microneedle 12, the third sidewall 123 of the microneedle 12 is approximately in the same plane as the base layer 11, or partially abuts against the base layer 11, such that the angle between the third sidewall 123 and the plane where the base layer 11 is located is 0° to ±10°, thereby completing the standing of the microneedle 12.

Furthermore, when the rotating mechanism includes the pressing plate 4211 and a microneedle patch 2 with an adhesion layer 21 needs to be prepared, or when the rotating mechanism includes the first roller 4212 and a microneedle patch 2 with an adhesion layer 21 needs to be prepared, the device for making the planar microneedle stands up also includes an adhesion layer applying structure.

The adhesion layer applying structure includes a gripping component and a visual recognizer. After the microneedle 12 stands up, the rotating mechanism is removed, and the gripping component uses the visual recognizer to correspondingly adhere the adhesion layer 21 to the side of the base layer 11 that is away from the microneedle 12. Subsequently, the rotating mechanism applies pressure to the adhesion layer 21, thereby securely adhering the adhesion layer 21 to the side of the base layer 11 that is away from the microneedle 12, as well as to the third sidewall 123 of the microneedle 12 or the protruding portion 127, thus forming the microneedle patch 2 with the adhesion layer 21.

The gripping component is preferably a vacuum suction gripping device.

More specifically, the adhesion layer applying structure further includes an adhesion layer transporter and a storage platform. The adhesion layer transporter is used for removing the protective film from the adhesion layer 21 and conveying the adhesion layer 21 to the storage platform. The storage platform is used to store the adhesion layer 21 and allows the gripping component to pick up the layers one by one. In the above, the protective film serves to prevent the adhesion layer 21 from adhering.

### Embodiment 6

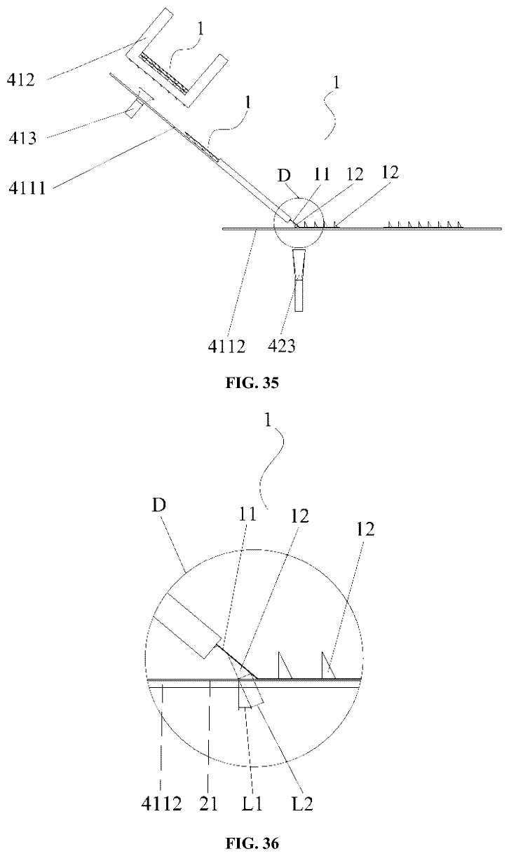

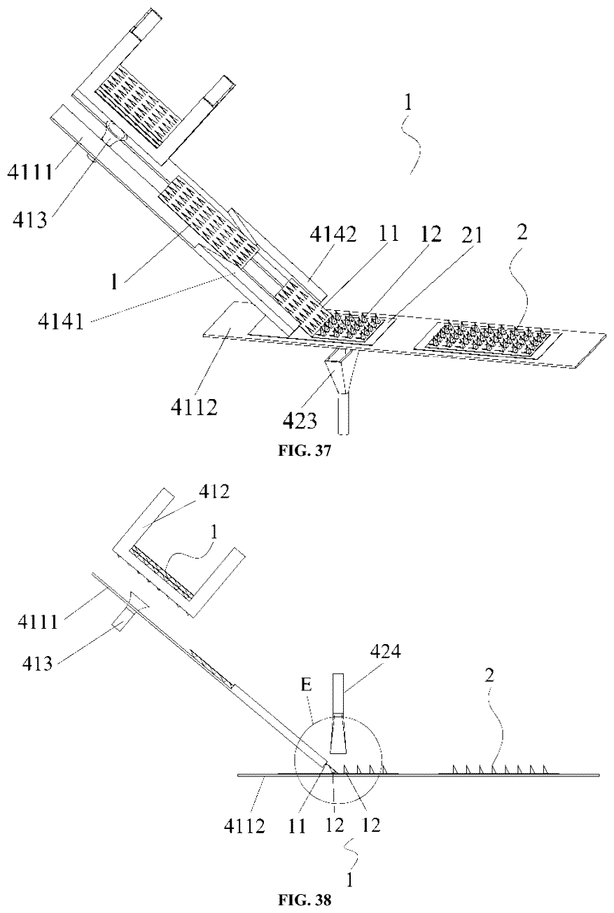

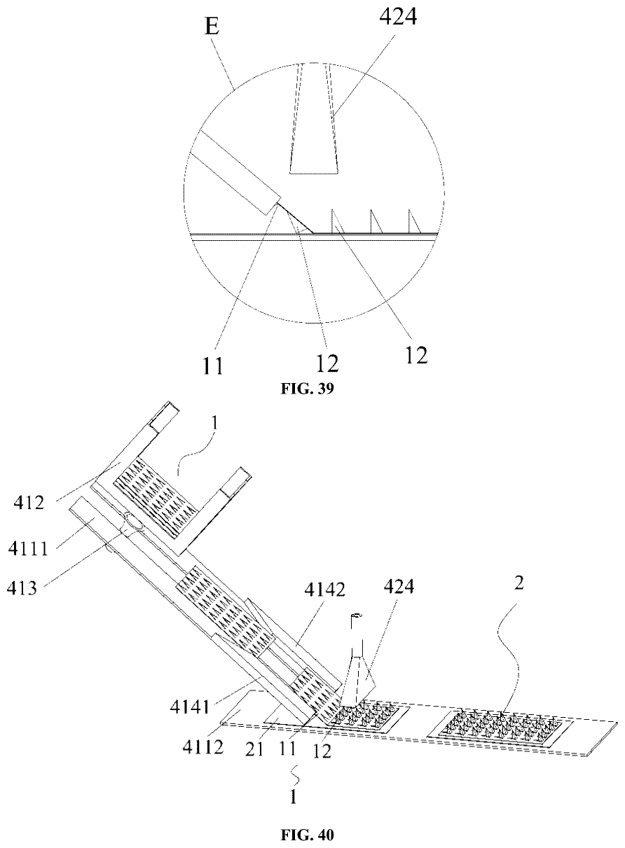

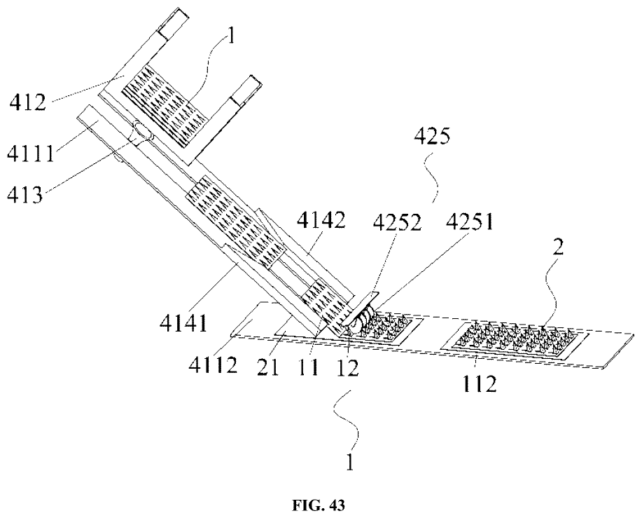

Referring to FIGS. 35-43, based on Embodiments 1-2, this embodiment also provides another type of planar microneedle standing device used to adhere the planar microneedle 1 to the adhesion layer 21, thereby forming the microneedle patch 2. The planar microneedle standing device includes a planar microneedle conveying assembly 41 and a planar microneedle rotating assembly 42.

The planar microneedle conveying assembly 41 is used to transport the planar microneedle 1 and the adhesion layer 21. The planar microneedle rotating assembly 42 is located at the junction between the planar microneedle 1 and the adhesion layer 21. The planar microneedle rotating assembly 42 is used for adhering the base layer 11 to the adhesion layer 21, rotating the first sidewall 121 of the microneedle 12, which is in the same plane as the base layer 11, until the third sidewall 123 is approximately in the same plane as the base layer 11 or partially abuts against the base layer 11, and make third sidewall 123 adhered to the adhesion layer 21, thereby completing the standing of the microneedle.

The planar microneedle conveying assembly 41 comprises a planar microneedle conveyor belt 4111 and a standing conveyor belt 4112. The planar microneedle conveyor belt 4111 is arranged inclinedly and is used for transporting the planar microneedle 1. The standing conveyor belt 4112 is provided horizontally and located at the lower end of the planar microneedle conveyor belt 4111. The standing conveyor belt 4112 is used to transport the adhesion layer 21, which has adhesive properties, and to receive the planar microneedle 1 from the planar microneedle conveyor belt 4111. The planar microneedle rotating assembly 42 is situated in close proximity to the lower end of the planar microneedle conveyor belt 4111.

During the standing process, the inclinedly provided planar microneedle conveyor belt 4111 is capable of transporting the planar microneedle 1 to the adhesion layer 21 in a manner that the planar microneedle 1 gradually contacts the adhesion layer 21 on the standing conveyor belt 4112, which ensures that the planar microneedle 1 and the adhesion layer 21 adhere smoothly to each other, without the presence of air bubbles or pocketing/hollowing which could cause wrinkling. The planar microneedle rotating assembly 42 is used for adhering the base layer 11 of the planar microneedle 1 to the adhesion layer 21 and rotating the microneedle 12 of the planar microneedle 1 around the junction between the third sidewall 123 and the base layer 11, from the state where the first sidewall 121 is in the same plane as the base layer 11 to the state where the third sidewall 123 is approximately in the same plane as the base later 11 or partially abuts against the base layer 11, thus achieving the standing of the microneedle 12. Subsequently, the bottom of the microneedle 12 adheres to the adhesion layer 21, and the planar microneedle 1 is combined with the adhesion layer 21 to form the microneedle patch 2.

This planar microneedle standing device is suitable for the two-step formation of the microneedle patch 2. Initially, the planar microneedle 1 is formed using a microneedle mold 32. Subsequently, the planar microneedle standing device adheres the planar microneedle 1 to the adhesion layer 21 and adjusts the microneedle 12 so that the third sidewall 123 is approximately in the same plane as the base layer 11, or partially abuts against the base layer 11 to thereby adhere to the adhesion layer 21.

Furthermore, the planar microneedle rotating assembly 42 applies a force to the planar microneedle 1 that is intended to adhere the planar microneedle 1 to the adhesion layer 21, with the direction of this force preferably being perpendicular to the conveying direction of the standing conveyor belt 4112. During operation, the force exerted by the planar microneedle rotating assembly 42 onto the planar microneedle 1 acts on the planar microneedle 1 row by row/gradually, enabling the base layer 11 of the planar microneedle 1 to adhere to the adhesion layer 21 row by row. As the planar microneedle 1 adheres to the adhesion layer 21 gradually/row by row, the microneedles 12 on the planar microneedle 1 experience a reaction force from the adhesion layer 21. Under the influence of this reaction force, each row of microneedles 12 can simultaneously rotate around the junctions between respective third sidewalls 123 and base layer 11, to make the first sidewall 121, which is in the same plane as the base layer 11, rotate until the third sidewall 123 is approximately in the same plane as the base layer 11 or partially abuts against the base layer 11, thereby ensuring that the third sidewall 123 adheres to the adhesion layer 21, completing the standing of the microneedle. Subsequently, the third sidewall 123 of the microneedle 12 adheres to the adhesion layer 21, resulting in the combination of the planar microneedle 1 and the adhesion layer 21 to form the microneedle patch 2. In this process, the base layer 11 and the adhesion layer 21 come into contact row by row, gradually achieving the bonding of the base layer 11 to the adhesion layer 21, allowing for the complete expulsion of air between the base layer 11 and the adhesion layer 21. Due to the angle between the planar microneedle conveyor belt 4111 and the standing conveyor belt 4112, the dimension of the third sidewall 123 of the microneedle 12 is comparable/close to the dimension of the projection thereof being projected onto the adhesion layer 21, and the third sidewall 123 of the microneedle 12 adheres to the adhesion layer 21 row by row, with the difference between the size L1 of the third sidewall 123 of the microneedle 12 and the dimension L2 of the projection thereof being projected onto the adhesion layer 21. Both the base layer 11 and the adhesion layer 21 possess a certain degree of elasticity, which helps to prevent wrinkling of the formed microneedle patch 2 caused by accumulation of allowance differences of the dimension differences when the third sidewalls 123 of all microneedles 12 are simultaneously adhered to the adhesion layer 21 during standing. Meanwhile, after the microneedle 12 rotates until the third sidewall 123 adheres to the adhesion layer 21, the adhesive force between the base layer 11 and the adhesion layer 21 enables the portion of the adhesion layer 21 in contact with the third sidewall 123 of the microneedle 12 to be smooth and wrinkle-free, improving the yield of the formed microneedle patch 2.

Further, at least one planar microneedle rotating assembly 42 is provided spaced along the conveying direction of the standing conveyor belt 4112, such that after the planar microneedle rotating assembly 42 near the planar microneedle conveyor belt 4111 adheres the planar microneedle 1 onto the adhesion layer 21, the remaining planar microneedle rotating assemblies 42 can continue to apply force to the microneedle patch 2 to enable more robust bonding between the planar microneedle 1 and the adhesion layer 21.

Further, the planar microneedle rotating assembly 42 can be of the following types.

Referring to FIGS. 35-37, the planar microneedle rotating assembly 42 may be a vacuum negative pressure adhesive component 423. The vacuum negative pressure adhesive component 423 is positioned beneath the standing conveyor belt 4112. The vacuum negative pressure adhesive component 423 is used to apply to the planar microneedle 1 a negative pressure airflow perpendicular to the conveying direction of the standing conveyor belt 4112. Under the influence of the suction force of this negative pressure airflow, the planar microneedle 1 is adhered to the adhesion layer 21. forming the microneedle patch 2.

At this time, grid-like through holes for allowing airflow to pass therethrough are provided on both the standing conveyor belt 4112 and the adhesion layer 21.

Referring to FIGS. 38-40, the planar microneedle rotating assembly 42 may be a wind pressure adhesive component 424. The wind pressure adhesive component 424 is provided above the standing conveyor belt 4112. The wind pressure adhesive component 424 can apply to the planar microneedle 1 an airflow perpendicular to the conveying direction of the standing conveyor belt 4112. Under the adhesion pressure of this airflow, the planar microneedle 1 is adhered to the adhesion layer 21 to form the microneedle patch 2.

At this time, the standing conveyor belt 4112 may be a mesh plane or a non-porous plane. and the adhesion layer 21 is a non-porous plane.

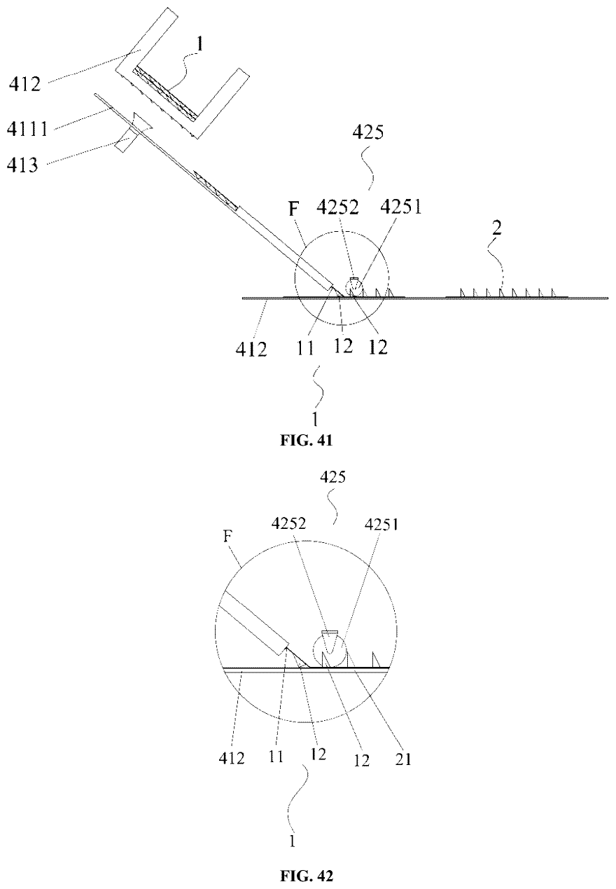

Referring FIGS. 41-43, the planar microneedle rotating assembly 42 may be a roller contact pressing component 425. The roller contact pressing component 425 includes at least one roller 4251 arranged at intervals, with the axial direction of at least one roller 4251 being perpendicular to the conveying direction of the standing conveyor 4112. Each roller 4251 is configured to roll on the base layer 11 between two adjacent microneedles 12 in each row of microneedles 12 on the planar microneedle 1, to gradually apply pressure row by row to the base layer 11, and cause the adhesion layer 21 to form a counterforce that is applied gradually row by row to the microneedles 12, thereby enabling the planar microneedles 1 to adhere to the adhesion layer 21 and form the microneedle patch 2. Due to the relatively small pressing area between the roller 4251 and the planar microneedle 1, the pressure intensity is greater, which enables a more secure adhesion between the base layer 11 and the adhesion layer 21, as well as between the bottoms of the microneedles 12 and the adhesion layer 21.

The roller contact pressing component 425 further includes a supporting stand 4252. where the roller 4251 is rotatably installed on the supporting stand 4252.

Preferably, the angle between the extending direction of the planar microneedle conveyor belt 4111 and the extending direction of the standing conveyor belt 4112 is 135°-179°. When the angle between the extending direction of the planar microneedle conveyor belt 4111 and the extending direction of the standing conveyor belt 4112 is less than 135°, it may easily result in the planar microneedle 1 adhering to the adhesion layer 21 at an excessively large angle between the planar microneedle 1 and the adhesion layer 21, resulting in increased horizontal component force parallel to the adhesion layer 21 and decreased vertical component force perpendicular to the adhesion layer 21, with the horizontal component force and the vertical component force being decomposed from the force which is acted on the planar microneedle 1 by the airflow applied to the planar microneedles 1 by the vacuum negative pressure adhesive component 423 and the wind pressure adhesive component 424, leading to: the inability to press the base layer 11 of the planar microneedles 1 onto the surface of the adhesion layer 21, resulting in that the base layer 11 cannot be attached and adhered to the adhesion layer 21, the adhesion force between the base layer 11 and the adhesion layer 21 is insufficient or the base layer 11 cannot be fully adhered to the adhesion layer 21. Due to the insufficient vertical component force acting on the planar microneedle 1, the base layer 11 cannot adhere or fully adhere to the adhesion layer 21, resulting in smaller counterforce for rotating the microneedles 12 generated by the adhesion layer 21 and causing the microneedles 12 to either not rotate or not fully rotate. When the roller contact pressing component 425 is pressing for making the microneedles stand up, the roller 4251 comes into contact with the planar microneedles 1 that are not transferred to the standing conveyor belt 4112 to damage the microneedles 12. When the angle between the extending direction of the planar microneedle conveyor belt 4111 and the extending direction of the standing conveyor belt 4112 is greater than 179°, the planar microneedle conveyor belt 4111 is almost parallel to the standing conveyor belt 4112, thus when the microneedles 12 are made to stand up, the bottoms of all the microneedles 12 adhere to the adhesion layer 21 simultaneously, where the cumulative value of the difference L1−L2 between the dimension L1 of the third sidewall of each of multiple microneedles 12 and the dimension L2 of the projection of the third sidewall being projected on the adhesion layer 21 becomes large, resulting in the formed microneedle patch 2 wrinkling and becoming a defective product.

Furthermore, the lower end of the planar microneedle conveyor belt 4111 is provided with a blocking plate 414 for allowing the planar microneedles 1 to pass therethrough, and the blocking plate 414 plays a role of guiding the conveying of the planar microneedles 1. The blocking plate 414 includes a first blocking plate 4141 and a second blocking plate 4142, with the first blocking plate 4141 and the second blocking plate 4142 being provided oppositely on two sides of the planar microneedle conveyor belt 4111.

Furthermore, the planar microneedle standing device further includes a storage device 412. The storage device 412 is located above the planar microneedle conveyor belt 4111 and positioned at the upper end of the planar microneedle conveyor belt 4111. The storage device 412 is used for storing the planar microneedles 1 that are vertically stacked.

The bottom of the storage device 412 is provided with a discharge port for discharging the planar microneedles 1. The discharge port is specifically embodied as having a shape similar to that of the planar microneedle 1 and having an area smaller than that of the planar microneedle 1, allowing it to store the planar microneedle 1. The discharge port is arranged parallel to the planar microneedle conveyor belt 4111, and the distance between the discharge port and the planar microneedle conveyor belt 4111 is greater than the thickness of the planar microneedle 1.

Further, the planar microneedle standing device further includes a planar microneedle adsorber 413. The planar microneedle adsorber 413 is arranged to penetrate through the planar microneedle conveyor belt 4111 and the planar microneedle adsorber 413 is positioned below the center of the planar microneedles 1 in the storage device 412. The planar microneedle adsorber 413 is capable of discharging a pulsed negative pressure airflow to adsorb the planar microneedles 1 layer by layer from the discharge port and lay them onto the planar microneedle conveyor belt 4111. The planar microneedle adsorber 413 can move along the axial direction and the planar microneedle adsorber 413 is able to pass through the planar microneedle conveyor belt 4111 to approach the bottom of the storage device 412 in order to discharge the pulsed negative pressure airflow. When the planar microneedles 1 are sucked from the storage device 412 onto the planar microneedle conveyor belt 4111, the planar microneedle adsorber 413 stops discharging the pulsed negative pressure airflow and retracts back along the axial direction to a position below the planar microneedle conveyor belt 4111. The specific process is as follows.

Since the planar microneedle adsorber 413 is positioned below the center of the planar microneedles 1 in the storage device 412, the adsorption force applied by the planar microneedle adsorber 413 to the planar microneedle 1 acts on the center of the planar microneedle 1. After deformation, the planar microneedle 1 is discharged from the discharge port. The center of the planar microneedle 1 first adheres to the planar microneedle conveyor belt 4111. Subsequently, the adsorption force creates a tensile force at the edge of the planar microneedle 1. Under the action of the tensile force, the edge of the planar microneedle 1 extends and lays flat on the planar microneedle conveyor belt 4111, thereby facilitating the transfer and lay flat of the planar microneedles 1 from the storage device 412 onto the planar microneedle conveyor belt 4111.

The planar microneedle standing device in the present disclosure has a simple structure, which is easy to process, and the standing method is fast and efficient.

In the description of the present specification, terms such as “one embodiment,” “some embodiments,” “embodiment,” “example,” “specific example,” or “some examples” refer to that specific features, structures, materials, or characteristics that are described in conjunction with that particular embodiment or example are encompassed in at least one embodiment or example of the present disclosure. In the description, that the use of these terms in an illustrative manner does not necessarily indicate that they refer to the same embodiment or example. Moreover, the specific features, structures, materials, or characteristics described may be combined in various suitable ways across any one or more embodiments or examples. Furthermore, those skilled in the art may combine and integrate different embodiments or examples described in this specification, along with the features of different embodiments or examples, as long as such combinations do not contradict one another.

While the embodiments of the present disclosure have been shown and described above, it is understood that these embodiments are exemplary and should not be construed as limiting the present disclosure. Those skilled in the art may make modifications, alterations, substitutions, and variations to the aforementioned embodiments within the scope of the present disclosure.

1. A planar microneedle, comprising a base layer and at least one microneedle rotatably arranged on the base layer, wherein each of the at least one microneedle comprises a needle tip, a needle base, and a middle part connecting the needle tip to the needle base, wherein a direction of the needle tip towards the needle base is aligned with an extending direction of the base layer; and the planar microneedle is formed by drying a filled raw material solution.

2. The planar microneedle according to claim 1, wherein the microneedle comprises a first sidewall, a circumferential sidewall, and a third sidewall; the first sidewall and the circumferential sidewall are positioned circumferentially around the microneedle, and the third sidewall is located at a bottom of the microneedle; the first sidewall and the base layer are located on a same plane, the third sidewall is connected to the base layer, and the microneedle is configured to rotate around a junction between the third sidewall and the base layer.

3. The planar microneedle according to claim 2, wherein the third sidewall comprises a first edge, wherein the first edge is connected to a side of the base layer away from the first sidewall; and the microneedle is configured to rotate around the first edge in a direction of the third sidewall towards the first sidewall, such that an angle between the third sidewall and a plane where the base layer is located is 0°-±10° and a base through-hole is formed at a junction between the microneedle and the base layer.

4. The planar microneedle according to claim 3, wherein the microneedle is configured to rotate around the first edge until the third sidewall partially abuts against a side of the base layer close to the microneedle, the third sidewall comprises a movable edge connected to the first edge, and an angle θ between the movable edge and a sidewall of the base through-hole is in a range of 0°<θ≤10°.

5. The planar microneedle according to claim 1, wherein a ratio of a maximum depth of the microneedle in a direction perpendicular to the base layer to a maximum length of the microneedle in a direction parallel to the base layer is 1:5 to 1:2.

6. A microneedle patch, comprising the planar microneedle according to claim 1.

7. (canceled)

8. A planar microneedle manufacturing device for manufacturing the planar microneedle according to claim 1, comprising a base, a mold, and a panel positioned above the base,
wherein a top of the base is provided with a recess configured for accommodating the mold;
a top of the mold is provided with multiple recessed forming grooves configured for forming microneedles;
the forming grooves each comprise a needle tip groove, a needle base groove, and a middle part groove, wherein the middle part groove communicates with the needle tip groove and the needle base groove, and a direction of the needle tip groove towards the needle base groove is a horizontal direction;
the panel is provided with a forming hole configured to accommodate all of the forming grooves of the mold, and the forming hole is configured for molding the base layer;
the top of the mold protrudes upward to form a separation portion, and the separation portion is arranged circumferentially around a sidewall of the forming groove, wherein when the mold is fitted into the recess of the base and the panel is pressed onto the tops of the base and the mold, a height of the separation portion is greater than or equal to a height of the panel.

9. The planar microneedle manufacturing device according to claim 8, wherein along the direction of the needle tip groove towards the needle base groove of the forming groove, a bottom wall of the forming groove extends outwards to form a protruding portion forming groove, and the protruding portion forming groove is configured for molding a protruding portion on the third sidewall of the microneedle.

10. The planar microneedle manufacturing device according to claim 8, wherein when a lower edge of a forming-groove bottom wall of the forming groove rotates around an upper edge of the forming-groove bottom wall until the forming-groove bottom wall abuts against the top of the mold, a length of the lower edge is greater than a length of a line connecting two intersection points of the lower edge with the forming groove.

11. A method for preparing a planar microneedle, using the planar microneedle manufacturing device according to claim 8, comprising following steps:
A1: assembling the base, the mold , and the panel;
A2: filling a drug-loaded solution into the needle tip groove of the mold and drying to form a needle tip of the microneedle; and
A3: filling a raw material solution into the middle part groove and the needle base groove of the mold, and the forming hole of the panel, and scraping and leveling the raw material solution along a top of the panel,
wherein the raw material solution in the forming hole dries to form the base layer, the raw material solution in the needle base groove of the mold dries to form the needle base of the microneedle, and the raw material solution in the middle part groove of the mold dries to form the middle part of the microneedle.

12. The method for preparing a planar microneedle according to claim 11, wherein step A3 further comprises:
filling the raw material solution into the forming hole of the panel, and then scraping and leveling the drug-loaded solution in the direction of the needle tip groove of the mold towards the needle base groove;
filling the raw material solution into the forming hole of the panel) until the forming hole is filled up, scraping and leveling the raw material solution along the top of the panel, and then vacuuming; or
filling the raw material solution into the forming hole of the panel until the forming hole is filled up, scraping and leveling the raw material solution along the top of the panel, and then centrifuging.

13. A planar microneedle standing device for making the planar microneedle according to claim 1 stand up, comprising a planar microneedle conveying assembly and a planar microneedle rotating assembly,
wherein the planar microneedle conveying assembly comprises a planar microneedle conveyor belt, and the planar microneedle conveyor belt is configured to convey the planar microneedle to the planar microneedle rotating assembly; and
the planar microneedle rotating assembly is configured to apply pressure to the at least one microneedle of the planar microneedle to rotate the at least one microneedle around the junction between the third sidewall and the base layer, so as to make the microneedle rotate to change from a state in which the first sidewall and the base layer are located in a same plane to a state in which an angle between the third sidewall and a plane where the base layer is located is 0°-±10°, thereby completing standing of the at least one microneedle.

14. The planar microneedle standing device according to claim 13, wherein the planar microneedle rotating assembly is a vacuum negative pressure adhesive component, and the vacuum negative pressure adhesive component is configured to apply a negative pressure airflow to the at least one microneedle from below the at least one microneedle;
the planar microneedle rotating assembly is a wind pressure adhesive component, and the wind pressure adhesive component is configured to apply airflow to the at least one microneedle from above the at least one microneedle; or
the planar microneedle rotating assembly is a roller contact pressing component, and the roller contact pressing component is configured to roll on the planar microneedle to apply pressure to the at least one microneedle.

15. The planar microneedle standing device according to claim 13, wherein the planar microneedle rotating assembly comprises a rotating mechanism and a microneedle accommodating mechanism, the rotating mechanism is a pressing plate; and
the rotating mechanism is configured to apply pressure to the at least one microneedle to make the at least one microneedle rotate around the first edge to be positioned within at least one accommodating groove at a top of the microneedle accommodating mechanism, thereby completing standing of the at least one microneedle.

16. The planar microneedle standing device according to claim 15, wherein a bottom of the pressing plate is provided with multiple pressure application portions, and the multiple pressure application portions are arranged in one-to-one correspondence with multiple accommodating grooves.

17. (canceled)

18. The planar microneedle standing device according to claim 14, wherein the planar microneedle rotating assembly comprises a rotating mechanism and a microneedle accommodating mechanism; the rotating mechanism comprises a first roller, the first roller is configured to apply pressure to the at least one microneedle to make the at least one microneedle rotate around the first edge to be positioned within at least one accommodating groove at a top of the microneedle accommodating mechanism, thereby completing standing of the at least one microneedle; and
the first roller and the microneedle accommodating mechanism are configured to move towards each other.

19. The planar microneedle standing device according to claim 18, wherein the rotating mechanism further comprises a second roller and a third roller; and
the second roller is wrapped with an adhesion layer, the second roller is configured to convey the adhesion layer to the first roller, the third roller is configured to be wrapped with and store a protective film obtained by tearing off the adhesion layer from the first roller, the first roller is configured to apply pressure to the at least one microneedle and adhere the adhesion layer to a side of the base layer away from the at least one microneedle and to the at least one microneedle.

20. The planar microneedle standing device according to claim 19, wherein a protective film peeling structure is arranged on a side of the first roller close to the third roller, wherein the protective film peeling structure is configured to control a position at which the protective film is peeled off from the adhesive base.

21. The planar microneedle standing device according to claim 15, wherein the microneedle accommodating mechanism further comprises multiple spaced fixing plate assemblies;
each of the fixing plate assemblies comprises a first clamping plate and a second clamping plate, wherein the first clamping plate is configured to move in a direction towards or away from the second clamping plate;
when the first clamping plate abuts against the second clamping plate, the accommodating groove is formed between the first clamping plate and the second clamping plate; and
when the microneedle is located within the accommodating groove, the first clamping plate and the second clamping plate clamp the microneedle.

22. (canceled)

23. The planar microneedle standing device according to claim 13, wherein the planar microneedle conveying assembly further comprises a standing conveyor belt; the planar microneedle conveyor belt is arranged inclinedly, and is configured to convey the planar microneedle, the standing conveyor belt is arranged horizontally and is positioned at a lower end of the planar microneedle conveyor belt, the standing conveyor belt is configured to convey a viscous adhesion layer or the microneedle accommodating mechanism and receive the planar microneedle from the planar microneedle conveyor belt; and
an angle between an extending direction of the planar microneedle conveyor belt and an extending direction of the standing conveyor belt is 135°-179°.
